(19) United States
(12) Patent Application Publication
(10) Pub. No.: US 2024/0238418 A1
(43) Pub. Date: Jul. 18, 2024

(54) CAR T CELL THERAPY METHOD

(71) Applicant: University of Florida Research Foundation, Inc., Gainesville, FL (US)

(72) Inventors: Elias Sayour, Newberry, FL (US); Jianping Huang, Gainesville, FL (US); Hector Ruben Mendez-Gomez, Gainesville, FL (US); Paul Antonio Castillo Caro, Gainesville, FL (US); Ruixuan Liu, Gainesville, FL (US)

(21) Appl. No.: 18/289,705

(22) PCT Filed: May 6, 2022
(86) PCT No.: PCT/US22/28136

§ 371 Date: Nov. 6, 2023

Related U.S. Application Data
(60) Provisional application No. 63/313,057, filed on Feb. 23, 2022.
(60) Provisional application No. 63/186,057, filed on May 7, 2021.

(51) Int. Cl. A61K 39/00; A61K 9/127; A61P 35/00

(52) U.S. Cl. CPC A61K 39/4611; A61K 9/1272; A61K 39/4631; A61K 39/464429; A61P 35/00; A61K 2239/39

(57) ABSTRACT

The disclosure provides a method of preconditioning a subject for chimeric antigen receptor (CAR) T cell therapy. The method comprises administering to the subject a composition comprising a nanoparticle comprising a positively-charged surface and an interior comprising (i) a core and (ii) at least two nucleic acid layers, wherein each nucleic acid layer is positioned between a cationic lipid bilayer, at least one day prior to administering CAR T cell therapy to the subject. The disclosure also provides a method of treating a solid tumor in a subject, the method comprising administering to a subject comprising a surface antigen negative solid tumor a first composition comprising the nanoparticle, wherein the nucleic acid within the nanoparticle encodes the surface antigen, and a second composition comprising a CAR T cell that targets the surface antigen.

### CROSS REFERENCE TO RELATED APPLICATIONS AND INCORPORATION BY REFERENCE

This application claims priority to U.S. Provisional Patent Application No. 63/186,057, filed May 7, 2021, and U.S. Provisional Patent Application No. 63/313,057, filed Feb. 23, 2022, the disclosures of which are hereby incorporated by reference in their entirety. The following applications also are hereby incorporated by reference in their entireties: International Patent Application No. PCT/US20/42606, filed Jul. 17, 2020; International Patent Application No. PCT/US21/16925, filed Feb. 5, 2021; and International Patent Application No. PCT/US21/18831, filed Feb. 19, 2021.

### GRANT FUNDING DISCLOSURE

This invention was made with government support under grant numbers K08 CA 199224 and R37 CA251978, awarded by the National Institutes of Health. The government has certain rights in the invention.

### FIELD OF THE INVENTION

This application relates to use of multilamellar nanoparticles to enhance treatment with chimeric antigen receptor T cells.

The Sequence Listing, which is a part of the present disclosure, is submitted concurrently with the specification as a text file. The name of the text file containing the Sequence Listing is “56528_Seqlisting.txt”, which was created on May 6, 2022 and is 1,881 bytes in size. The subject matter of the Sequence Listing is incorporated herein in its entirety by reference.

### BACKGROUND

Chimeric antigen receptor (CAR) T cells are T lymphocytes genetically engineered to express a receptor that recognizes a particular cell surface antigen, such as a cancer cell antigen. The promise of CAR T cell therapy has been realized for blood borne cancers, such leukemia, lymphoma, and multiple myeloma. However, CAR T cell therapy is still in its infancy and, despite numerous advancements in the underlying technology, significant obstacles to widespread use remain. For example, CAR-T cell therapy has yet to be successful for solid tumors. CAR T cells' poor ability to traffic to and infiltrate solid tumors is a significant challenge, and the tumor microenvironment is largely immunosuppressive. Regulatory T cells, myeloid-derived suppressor cells, and tumor-associated macrophages (TAMs), among others, express cell surface ligands (e.g., CD80/CD86) that bind inhibitory receptors on T cells (e.g., CTLA-4), as well as secrete soluble factors that suppress or trigger apoptosis in T cells. Another significant barrier to the effectiveness of CAR T cell therapy against solid is surface antigen heterogeneity or lack of expression of surface antigen within the solid tumor. These challenges have impeded progress in applying CAR T cell therapy to a large percentage of cancer patients, those with solid tumors.

Additionally, conditioning therapy is generally required prior to CAR T cell administration. Lymphodepletion (LD) conditioning prior to CAR T cell administration is believed to create a “favorable” environment for CAR T cell expansion and survival by eliminating regulatory T cells. LD conditioning often involves administration of chemotherapeutic agents, such as cyclophosphamide, fludarabine, pentostatin, or bendamustine, or total body irradiation. A cycle of LD conditioning, e.g., chemotherapy administration over the course of one to five days, is usually administered two to 14 days prior to the infusion of the CAR T cells to allow “space” for the CAR T cells to proliferate and activate. All CAR T cell therapeutics currently approved by the U.S. Food and Drug Administration require chemotherapy-based lymphodepletion conditioning therapy prior to infusion of the CAR T cell product. While lymphodepletion conditioning therapy is tolerated by patients, it is often associated with significant side effects and puts patients at significant risk of infection.

There remains a need for CAR T cell therapeutic regimens which, e.g., improve efficacy, expand patient populations that benefit from CAR T cell therapy, and minimize unwanted side effects.

### SUMMARY

The disclosure provides a method of preconditioning a subject for chimeric antigen receptor (CAR) T cell therapy. The method comprises administering to the subject a first composition comprising a nanoparticle comprising a positively-charged surface and an interior comprising (i) a core and (ii) at least two nucleic acid layers, wherein each nucleic acid layer is positioned between a cationic lipid bilayer. The composition is administered to the subject at least one day prior to administering CAR T cell therapy to the subject. In various aspects, the method further comprises administering CAR T cell therapy to the subject. Optionally, the first composition is administered between two and 14 days (e.g., about five to about eight days, such as seven days) prior to administering the CAR T cell therapy to the subject. Also, in various aspects of the disclosure, the subject is not administered lymphodepletion therapy within 21 days prior to administration of the CAR T cell therapy. In exemplary embodiments, the nanoparticle comprises at least three nucleic acid layers (e.g., at least four or at least five nucleic acid layers), each of which is positioned between a cationic lipid bilayer. In various aspects, the outermost layer of the nanoparticle comprises a cationic lipid bilayer. In various instances, the surface comprises a plurality of hydrophilic moieties of the cationic lipid of the cationic lipid bilayer. In exemplary aspects, the core comprises a cationic lipid bilayer. Optionally, the core comprises less than about 0.5 wt % nucleic acid. The diameter of the nanoparticle, in various aspects, is about 50 nm to about 250 nm in diameter, optionally, about 70 nm to about 200 nm in diameter. In exemplary instances, the nanoparticle is characterized by a zeta potential of about +40 mV to about +60 mV, optionally, about +45 mV to about +55 mV. The nanoparticle, in various instances, has a zeta potential of about 50 mV. In some aspects, the nucleic acid molecules are present at a nucleic acid molecule:cationic lipid ratio of about 1 to about 5 to about 1 to about 25, optionally, about 1 to about 15, about 1 to about 10 or about 1 to about 7.5. In various aspects, the nucleic acid molecules are RNA molecules, optionally, messenger RNA (mRNA). In various aspects, the mRNA is in vitro transcribed mRNA wherein the in vitro transcription template is cDNA made from RNA extracted from a tumor cell. In various aspects, the nanoparticle comprises a mixture of RNA, such as RNA isolated from a tumor of a human. In various aspects, the nucleic acid does not encode the tumor antigen recognized by the CAR T cell. In various aspects, the subject is suffering from a solid tumor, such as a glioblastoma, medulloblastoma, diffuse intrinsic pontine glioma, a peripheral tumor with metastatic infiltration into the central nervous system, or osteosarcoma.

The disclosure further contemplates use of a first composition comprising a nanoparticle comprising a positively-charged surface and an interior comprising (i) a core and (ii) at least two nucleic acid layers, wherein each nucleic acid layer is positioned between a cationic lipid bilayer, for preconditioning a subject for CAR T cell therapy, wherein the composition is administered to the subject at least one day prior to administering CAR T cell therapy to the subject. Use of the nanoparticle in the preparation of a medicament for preconditioning a subject for CAR T cell therapy also is contemplated, as is the nanoparticle composition described herein for use in preconditioning a subject for CAR T cell therapy.

The disclosure also provides a method of treating a solid tumor in a subject. The method comprises administering to a subject comprising a surface antigen negative solid tumor a first composition comprising a nanoparticle comprising a positively-charged surface and an interior comprising (i) a core and (ii) at least two nucleic acid layers, wherein each nucleic acid layer is positioned between a cationic lipid bilayer and the nucleic acid encodes the surface antigen. A second composition comprising a T cell expressing a chimeric antigen receptor (CAR) that targets the surface antigen also is administered to the subject. Optionally, the first composition is administered at least one day prior the second composition comprising the CAR T cells. Also, in various aspects of the disclosure, the subject is not administered lymphodepletion therapy within 21 days prior to administration of the CAR T cell therapy. In exemplary embodiments, the nanoparticle comprises at least three nucleic acid layers (e.g., at least four or at least five nucleic acid layers), each of which is positioned between a cationic lipid bilayer. In various aspects, the outermost layer of the nanoparticle comprises a cationic lipid bilayer. In various instances, the surface comprises a plurality of hydrophilic moieties of the cationic lipid of the cationic lipid bilayer. In exemplary aspects, the core comprises a cationic lipid bilayer. Optionally, the core comprises less than about 0.5 wt % nucleic acid. The diameter of the nanoparticle, in various aspects, is about 50 nm to about 250 nm in diameter, optionally, about 70 nm to about 200 nm in diameter. In exemplary instances, the nanoparticle is characterized by a zeta potential of about +40 mV to about +60 mV, optionally, about +45 mV to about +55 mV. The nanoparticle, in various instances, has a zeta potential of about 50 mV. In some aspects, the nucleic acid molecules are present at a nucleic acid molecule:cationic lipid ratio of about 1 to about 5 to about 1 to about 25, optionally, about 1 to about 15, about 1 to about 10 or about 1 to about 7.5. In various aspects, the nucleic acid molecules are RNA molecules, optionally, messenger RNA (mRNA). In various aspects, the solid tumor is present in lung, liver, bone, spleen, or lymph node. An exemplary solid tumor is osteosarcoma. In various aspects, the surface antigen is CD70, and the CAR T cell expresses a CAR which binds CD70.

The disclosure also provides use of a first composition comprising a nanoparticle comprising a positively-charged surface and an interior comprising (i) a core and (ii) at least two nucleic acid layers, wherein each nucleic acid layer is positioned between a cationic lipid bilayer and the nucleic acid encodes the surface antigen and a second composition comprising a CAR T cell that targets the surface antigen for treating a surface antigen negative solid tumor in a subject. Optionally, the first composition is administered at least one day prior the second composition (comprising CAR T cells). Use of the nanoparticle in the preparation of a medicament for treating a surface antigen negative solid tumor with CAR T cell therapy also is contemplated, as is the nanoparticle composition described herein for use in treating a surface antigen negative solid tumor with CAR T cell therapy.

Additional embodiments and aspects of the presently disclosed nanoparticles, pharmaceutical compositions, and methods are provided below.

### BRIEF DESCRIPTION OF THE DRAWINGS

FIG. 1A is a series of illustrations of a lipid bilayer, liposome and a general scheme leading to multilamellar (ML) RNA NPs (boxed).

FIG. 1B is a pair of CEM images of uncomplexed NPs (left) and ML RNA NPs (right).

FIG. 2A is an illustration of a general scheme leading to cationic RNA lipoplexes.

FIG. 2B is an illustration of a general scheme leading to cationic RNA lipoplexes.

FIG. 2C is a CEM image of uncomplexed NPs, FIG. 2D is a CEM image of RNA LPXs, and FIG. 2E is a CEM image of ML RNA NPs.

FIG. 2F is a graph of the % CD86+ of CD11c+MHC Class II+ splenocytes present in the spleens of mice treated with ML RNA NPs (ML RNA-NPs), RNA LPXs, anionic LPXs, or of untreated mice.

FIG. 2G is a graph of the % CD44+CD62L+ of CD8+ splenocytes present in the spleens of mice treated with ML RNA NPs (ML RNA-NPs), RNA LPXs, anionic LPXs, or of untreated mice.

FIG. 2H is a graph of the % CD44+CD62L of CD4+ splenocytes present in the spleens of mice treated with ML RNA NPs (ML RNA-NPs), RNA LPXs, anionic LPXs, or of untreated mice.

FIG. 2I is a graph of the % survival of mice treated with ML RNA NPs (ML RNA-NPs), RNA LPXs, anionic LPXs, or of untreated mice.

FIG. 2J is a graph of the amount of IFN-α produced in mice upon treatment with ML RNA NPs (ML RNA-NPs), RNA LPXs, anionic LPXs, or of untreated mice.

FIG. 3A is a graph of the % expression of CD8 or CD44 and CD8 of CD3+ cells plotted as a function of time post administration of ML RNA NPs.

FIG. 3B is a graph of the % expression of PDL1, MHC II, CD86 or CD80 of CD11c+ cells plotted as a function of time post administration of ML RNA NPs.

FIG. 3C is a graph of the % expression of CD44 and CD8 of CD3+ cells plotted as a function of time post administration of ML RNA NPs.

FIG. 3D is a graph of the % survival of canines treated with ML RNA NPs compared to the median survival (dotted line).

FIG. 3E illustrates the percentage of lymphocytes (y-axis) elicited post-administration of ML RNA-NPs (x-axis) in a canine model.

FIG. 3F illustrates interferon-α production (pg/mL; y-axis) in the hours following administration of ML RNA-NPs in a canine model.

FIG. 3G illustrates an increase in CD80+ expression on Cd11c+ cells (% expression, y-axis) in the hours following administration of the ML RNA-NPs (x-axis).

FIG. 3H illustrates expression of CD8 and CD44+CD8+ cells in the hours following administration of the ML RNA-NPs (x-axis) to canine subject.

FIG. 4A is a timeline of the long-term survivor treatment. First and second tumor inoculations are shown. FIG. 4B is a graph of the percent survival of animals after the second tumor inoculation for each of the three groups of mice: two groups treated before 2^{nd} tumor inoculation with ML RNA NPs comprising non-specific RNA (RNA not specific to the tumor in the subject; Green Fluorescence Protein (GFP) or pp65) and one group treated before 2^{nd} tumor inoculation with ML RNA NPs comprising tumor specific RNA or untreated animals prior to 2^{nd} tumor inoculation. Control group survival percentage is noted as “Untreated”.

FIG. 5 is a graph of the percentage of surviving mice of a group treated with ML RNA NPs alone (RNA-NP) or in combination with PDL1 monoclonal antibodies (RNA-NP+PDL1 mAb) as a function of time (days) after tumor implantation. Control groups included untreated mice (Untreated), mice treated with ML NPs without any RNA (NP Alone), and mice treated with PDL1 monoclonal antibodies alone (PDL1 mAb). *p<0.05, Gehan-Breslow-Wilcox.

FIGS. 6A-6C are line graphs illustrating tumor volume (mm³) of melanoma (FIG. 6A), percent survival in a sarcoma model (FIG. 6B), and percent survival in a metastatic lung model (FIG. 6C) at various days post-tumor implantation. The figures demonstrate that the ML RNA-NPs of the disclosure mediate effective anti-tumor immune responses against immunologically cold tumors in vivo.

FIGS. 7A-7C demonstrate that non-specific ML RNA-NPs of the disclosure mediate significant anti-tumor efficacy. FIG. 7A: Tumor volumes (mm³) of C57Bl/6 mice (7-8/group) bearing subcutaneous B16F0 tumors were vaccinated with luciferase RNA-NPs once weekly (×3) or treated twice weekly with PD-L1-mAbs (×3). FIG. 7B: Survival plot (% survival; y-axis) of BALB/c mice (8/group) inoculated with K7M2 lung tumors and vaccinated with three weekly GFP RNA-NPs (×3) or twice weekly PD-L1 mAbs. FIG. 7C: Non-specific RNA-NPs (luciferase) sensitize response to ICIs (immune checkpoint inhibitor) in a checkpoint resistant murine tumor model (B16F0). Tumor volumes (mm³) provided on y-axis; days after tumor implantation provided on x-axis.

FIGS. 8A and 8B: RNA-NPs sensitize response to CAR T cells. FIG. 8A is a line graph illustrating tumor size (y-axis, fluorescence as a surrogate for tumor size) at various days after tumor implantation (x-axis). Subjects were irradiated (5 Gy) 24 hours before CAR T cell administration. This study utilized KR158 cells expressing CD70 (CD70KR158) prior to implantation. In this RNA-NP resistant tumor model (CD70KR158), substantial synergy was observed when CD70 CAR T cells (1×10⁷ administered) were administered with nanoparticles comprising nucleic acid encoding CD70. RNA-NPs (encoding CD70) were administered weekly, beginning 24 h after CAR T cell infusion (p=0.05). FIG. 8B is a bar graph illustrating the number of CAR T cells (y-axis) in peripheral blood with and without RNA-NP co-therapy (x-axis). At 6 h post-administration of RNA-NP, RNA-NPs induced CD70 CAR T cell mobilization out of peripheral blood (*, p=0.0179).

FIGS. 9A-9C: RNA-NPs elicit IFN-α surge, activation of peripheral DCs, and margination of lymphocytes in only 2 h after infusion in canines with terminal malignancies. FIG. 9A is a line graph illustrating IFN-α levels (pg/mL, y-axis) at various timepoints (pre-administration of RNA-NPs, two hours post-administration, and six hours post-administration; x-axis). FIG. 9B is a line graph illustrating % CD80+ dendritic cells (y-axis) at various timepoints (pre-administration of RNA-NPs, two hours post-administration; x-axis). FIG. 9C is a line graph illustrating absolute lymphocyte count (K/μL; y-axis) at various timepoints (pre-administration of RNA-NPs, two hours post-administration, six hours post-administration, one week post-administration, two weeks post-administration, and six weeks post-administration; x-axis).

FIGS. 10A and 10B: In vitro antitumor specific killing. FIG. 10A shows antitumor specific IFN-γ release after co-culture of CD70+ tumor and CD70-directed CAR T cells in K7M2 OSA murine solid tumor model (two-way ANOVA, Turkey's multiple comparisons). The K7M2 cells expressed CD70 prior to the study. FIG. 10B shows antitumor specific killing that correlates with increasing CAR T doses.

FIG. 11: In a surface antigen negative tumor model (K7M2 cells, which did not express CD70 prior to implantation), administration of RNA-NPs comprising nucleic acid that encodes CD70 sensitized the solid tumor to CD70 CAR T cells. RNA-NPs (i.v.) were administered on day 5 after K7M2 tail vein inoculation, day 7, after CAR T administration, and weekly thereafter (×3)-(8/group; p=0.03). “WT-NPs” comprise CD70-negative total tumor-derived mRNA. Untreated subjects and subjects administered CD70 CAR T cells survived less than 30 days. Administration of WT-NPs, NPs encoding CD70, and WT-NPs in combination with the CD70 CAR T cells extended survival. Remarkably, administration of NPs encoding CD70 in combination with CD70 CAR T cells significantly improved survival well beyond the other treatments provided.

FIGS. 12A and 12B are line graphs demonstrating % of CD70+ splenocytes (FIG. 12A) and % of CD70+ liver cells (FIG. 12B) at 36 hours after injection of nanoparticles of the disclosure comprising RNA encoding CD70 (5 micrograms and 25 micrograms). Mice were injected with RNA-NPs and organs (spleen and liver) were collected to characterize CD70 expression. Systemic administration of NP resulted in antigen expression in splenocytes and liver.

FIGS. 13A and 13B illustrate results of transduction studies and in vitro killing of tumor cells. FIG. 13A includes plots demonstrating expression of CD70 in dendritic cells (DC2.4) and brain tumor cells (KR158) following application of CD70-encoding NP. DC2.4 and KR158 cells do not naturally express CD70; about 73% of DC2.4 cells and about 95% of KR158 cells expressed CD70 after exposure to the NP. FIG. 13B is a line graph illustrating the effect of NP comprising non-specific RNA (RNA encoding ovalbumin (OVA)), NP comprising RNA encoding CD70, a combination of OVANP with CD70-targeted CAR T cells, and a combination of CD70NP with CD70-targeted CAR T cells on tumor cell viability. KR158 cells, which do not naturally express CD70, were utilized in this in vitro assay, which measured luminescence as a surrogate for tumor cell viability (y-axis). The ratio of effector cell (CAR T cell) to target cell (tumor) is noted on the x-axis. The combination of CD70 CAR T cell and NP encoding CD70 mediated a significant reduction in viable tumor cells (i.e., the combination resulted in significantly more tumor cell death than the other treatments).

### DETAILED DESCRIPTION

The disclosure provides materials and methods for, e.g., improving the efficacy of chimeric antigen receptor (CAR) T cell therapy and/or expanding the patient population that responds to the therapy. For example, the disclosure provides a method of preconditioning a subject for chimeric antigen receptor (CAR) T cell therapy. The method comprises administering to the subject a first composition comprising a nanoparticle (NP) comprising a positively-charged surface and an interior comprising (i) a core and (ii) at least two nucleic acid layers, wherein each nucleic acid layer is positioned between a cationic lipid bilayer. The composition is administered to the subject at least one day prior to administering CAR T cell therapy to the subject. Previously, it was generally believed that lymphodepletion (LD) conditioning was required to achieve optimal T cell expansion and activation in vivo. LD conditioning serves to wipe out a subject's lymphocyte population; such “negative conditioning” is associated with unwanted side effects, such as increased susceptibility for infection, low blood count, nausea, vomiting, fatigue, and hair loss. It was surprisingly determined that administration of the nanoparticle composition described herein sufficiently primes the body to accept CAR T cell therapy such that LD conditioning is not required. This “positive conditioning” avoids the unwanted side effects associated with LD therapy. Further, administration of the nanoparticle composition described herein creates an immunological milieu that promotes T cell trafficking to solid tumors and enhanced activation in the otherwise immunosuppressive tumor microenvironment. The observations described herein represent a paradigm shift in preparing subjects for CAR T cell treatment.

The disclosure also provides a method of treating a solid tumor in a subject, wherein the solid tumor is “surface antigen negative,” meaning that the tumor does not express sufficient level of surface tumor antigen to be clinically responsive to CAR T cell therapy prior to treatment as disclosed herein. The instant method comprises administering to the subject comprising a surface antigen negative solid tumor a first composition comprising a nanoparticle comprising a positively-charged surface and an interior comprising (i) a core and (ii) at least two nucleic acid layers, wherein each nucleic acid layer is positioned between a cationic lipid bilayer and the nucleic acid encodes the surface antigen. The method further comprises administering a second composition comprising a T cell expressing a chimeric antigen receptor (CAR) (i.e., a CAR T cell) that targets the surface antigen. It has been determined that administration of nanoparticles described herein which contain mRNA encoding a surface antigen targeted by a CAR T cell can transform a refractory solid tumor (i.e., a tumor which does not respond to CAR T cell therapy due to lack of sufficient levels of surface antigen expression) into a tumor which responds to CAR T cell therapy. The materials and methods described herein unlock the potential of CAR T cell therapy for new patient populations (those with refractory solid tumors) as well as potentially enable use of “off the shelf” CAR T cell therapies.

Various aspects of the method are described below. The use of section headings is merely for the convenience of reading; it should be understood that the disclosure should be read as a whole and all combinations of features described herein are contemplated.

### Nanoparticles

The nanoparticles of the method comprise a cationic lipid and nucleic acids. As used herein the term “nanoparticle” refers to a particle that is less than about 1000 nm in diameter. As the nanoparticles of the present disclosure comprise cationic lipids that have been processed to induce liposome formation, the presently disclosed nanoparticles in various aspects comprise liposomes. Liposomes are artificially-prepared vesicles which, in exemplary aspects, are primarily composed of a lipid bilayer. Liposomes in various instances are used as a delivery vehicle for the administration of nutrients and pharmaceutical agents. In various aspects the liposomes of the present disclosure are of different sizes and the composition may comprise one or more of (a) a multilamellar vesicle (MLV) which may be hundreds of nanometers in diameter and may contain a series of concentric bilayers separated by narrow aqueous compartments, (b) a small unicellular vesicle (SUV) which may be smaller than, e.g., 50 nm in diameter, and (c) a large unilamellar vesicle (LUV) which may be between, e.g., 50 and 500 nm in diameter. Liposomes in various instances are designed to comprise opsonins or ligands in order to improve the attachment of liposomes to unhealthy tissue or to activate events such as, but not limited to, endocytosis. In exemplary aspects, liposomes contain a low or a high pH in order to improve the delivery of the pharmaceutical formulations. In various instances, liposomes are formulated depending on the physicochemical characteristics such as, but not limited to, the pharmaceutical formulation entrapped and the liposomal ingredients, the nature of the medium in which the lipid vesicles are dispersed, the effective concentration of the entrapped substance and its potential toxicity, any additional processes involved during the application and/or delivery of the vesicles, the optimization size, polydispersity and the shelf-life of the vesicles for the intended application, and the batch-to-batch reproducibility and possibility of large-scale production of safe and efficient liposomal products.

In exemplary embodiments, the nanoparticle comprises a surface and an interior comprising (i) a core and (ii) at least two nucleic acid layers, optionally, more than two nucleic acid layers. In exemplary instances, each nucleic acid layer is positioned between a lipid layer, e.g., a cationic lipid layer. In exemplary aspects, the nanoparticles are multilamellar comprising alternating layers of nucleic acid and lipid. In exemplary embodiments, the nanoparticle comprises at least three nucleic acid layers, each of which is positioned between a cationic lipid bilayer. In exemplary aspects, the nanoparticle comprises at least four or five nucleic acid layers, each of which is positioned between a cationic lipid bilayer. In exemplary aspects, the nanoparticle comprises at least more than five (e.g., 6, 7, 8, 9, 10, 11, 12, 13, 14, 15, 16, 17, 18, 19, 20, or more) nucleic acid layers, each of which is positioned between a cationic lipid bilayer. As used herein the term “cationic lipid bilayer” is meant a lipid bilayer comprising, consisting essentially of, or consisting of a cationic lipid or a mixture thereof. Suitable cationic lipids are described herein. As used herein the term “nucleic acid layer” is meant a layer of the presently disclosed nanoparticle comprising, consisting essentially of, or consisting of a nucleic acid, e.g., RNA.

The unique structure of the nanoparticle of the present disclosure results in mechanistic differences in how the multilamellar nanoparticles (ML-NPs) exert a biological effect. Previously described RNA-based nanoparticles exert their effect, at least in part, through the toll-like receptor 7 (TLR7) pathway. Surprisingly, the multi-lamellar nanoparticles of the instant disclosure mediate efficacy independent of TLR7. While not wishing to be bound to any particular theory, intracellular pathogen recognition receptors (PRRs), such as MDA-5, appear more relevant to biological activity of the multi-lamellar nanoparticles than TLRs. This likely allows ML RNA-NPs to stimulate multiple intracellular PRRs (e.g., RIG-I, MDA-5) as opposed to singular TLRs (e.g., TLR7 in the endosome) culminating in greater release of type I interferons and induction of more potent innate immunity. This allows RNA-NPs to demonstrate superior efficacy with long-term survivor benefit.

In various aspects, the presently disclosed nanoparticle comprises a positively-charged surface. In some instances, the positively-charged surface comprises a lipid layer, e.g., a cationic lipid layer. In various aspects, the outermost layer of the nanoparticle comprises a cationic lipid bilayer. Optionally, the cationic lipid bilayer comprises, consists essentially of, or consists of DOTAP. In various instances, the surface comprises a plurality of hydrophilic moieties of the cationic lipid of the cationic lipid bilayer. In some aspects, the core comprises a cationic lipid bilayer. In various instances, the core lacks nucleic acids, optionally, the core comprises less than about 0.5 wt % nucleic acid.

In exemplary aspects, the nanoparticle has a diameter within the nanometer range and accordingly in certain instances are referred to herein as “nanoliposomes” or “liposomes”. In exemplary aspects, the nanoparticle has a diameter between about 50 nm to about 500 nm, e.g., about 50 nm to about 450 nm, about 50 nm to about 400 nm, about 50 nm to about 350 nm, about 50 nm to about 300 nm, about 50 nm to about 250 nm, about 50 nm to about 200 nm, about 50 nm to about 150 nm, about 50 nm to about 100 nm, about 100 nm to about 500 nm, about 150 nm to about 500 nm, about 200 nm to about 500 nm, about 250 nm to about 500 nm, about 300 nm to about 500 nm, about 350 nm to about 500 nm, or about 400 nm to about 500 nm. In exemplary aspects, the nanoparticle has a diameter between about 50 nm to about 300 nm, e.g., about 100 nm to about 250 nm, about 110 nm±5 nm, about 115 nm±5 nm, about 120 nm±5 nm, about 125 nm±5 nm, about 130 nm±5 nm, about 135 nm±5 nm, about 140 nm±5 nm, about 145 nm±5 nm, about 150 nm±5 nm, about 155 nm±5 nm, about 160 nm±5 nm, about 165 nm±5 nm, about 170 nm±5 nm, about 175 nm±5 nm, about 180 nm±5 nm, about 190 nm±5 nm, about 200 nm±5 nm, about 210 nm±5 nm, about 220 nm±5 nm, about 230 nm±5 nm, about 240 nm±5 nm, about 250 nm±5 nm, about 260 nm±5 nm, about 270 nm±5 nm, about 280 nm±5 nm, about 290 nm±5 nm, or about 300 nm±5 nm. In exemplary aspects, the nanoparticle is about 50 nm to about 250 nm in diameter. In some aspects, the nanoparticle is about 70 nm to about 200 nm in diameter.

In exemplary aspects, the nanoparticle is present in a pharmaceutical composition comprising a heterogeneous mixture of nanoparticles ranging in diameter, e.g., about 50 nm to about 500 nm or about 50 nm to about 250 nm in diameter. Optionally, the pharmaceutical composition comprises a heterogeneous mixture of nanoparticles ranging from about 70 nm to about 200 nm in diameter.

In exemplary instances, the nanoparticle is characterized by a zeta potential of about +40 mV to about +60 mV, e.g., about +40 mV to about +55 mV, about +40 mV to about +50 mV, about +40 mV to about +50 mV, about +40 mV to about +45 mV, about +45 mV to about +60 mV, about +50 mV to about +60 mV, about +55 mV to about +60 mV. In exemplary aspects, the nanoparticle has a zeta potential of about +45 mV to about +55 mV. The nanoparticle in various instances, has a zeta potential of about +50 mV. In various aspects, the zeta potential is greater than +30 mV or +35 mV. The zeta potential is one parameter which distinguishes the nanoparticles of the present disclosure and those described in Sayour et al., Oncoimmunology 6(1): e1256527 (2016).

In exemplary embodiments, the nanoparticles comprise a cationic lipid. In some embodiments, the cationic lipid is a low molecular weight cationic lipid such as those described in U.S. Patent Application Publication No. 20130090372, the contents of which are herein incorporated by reference in their entirety. The cationic lipid in exemplary instances is a cationic fatty acid, a cationic glycerolipid, a cationic glycerophospholipid, a cationic sphingolipid, a cationic sterol lipid, a cationic prenol lipid, a cationic saccharolipid, or a cationic polyketide. In exemplary aspects, the cationic lipid comprises two fatty acyl chains, each chain of which is independently saturated or unsaturated. In some instances, the cationic lipid is a diglyceride. For example, in some instances, the cationic lipid may be a cationic lipid of Formula I or Formula II:

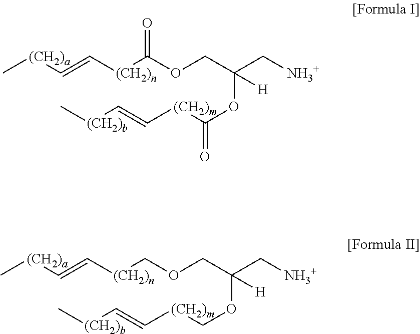

wherein each of a, b, n, and m is independently an integer between 2 and 12 (e.g., between 3 and 10). In some aspects, the cationic lipid is a cationic lipid of Formula I wherein each of a, b, n, and m is independently an integer selected from 3, 4, 5, 6, 7, 8, 9, and 10. In exemplary instances, the cationic lipid is DOTAP (1,2-dioleoyl-3-trimethylammonium-propane), or a derivative thereof. In exemplary instances, the cationic lipid is DOTMA (1,2-di-O-octadecenyl-3-trimethylammonium propane), or a derivative thereof.

In some embodiments, the nanoparticles comprise liposomes formed from 1,2-dioleyloxy-N, N-dimethylaminopropane (DODMA) liposomes, DiLa2 liposomes from Marina Biotech (Bothell, Wash.), 1,2-dilinoleyloxy-3-dimethylaminopropane (DLin-DMA), 2,2-dilinoleyl-4-(2-dimethylaminoethyl)-[1,3]-dioxolane (DLin-KC2-DMA), and MC3 (US20100324120; herein incorporated by reference in its entirety). In some embodiments, the nanoparticles comprise liposomes formed from the synthesis of stabilized plasmid-lipid particles (SPLP) or stabilized nucleic acid lipid particle (SNALP) that have been previously described and shown to be suitable for oligonucleotide delivery in vitro and in vivo. The nanoparticles in some aspects are composed of 3 to 4 lipid components in addition to the nucleic acid molecules. In exemplary aspects, the liposome comprises 55% cholesterol, 20% disteroylphosphatidyl choline (DSPC), 10% PEG-S-DSG, and 15% 1,2-dioleyloxy-N,N-dimethylaminopropane (DODMA), as described by Jeffs et al., Pharm Res. 2005; 22(3):362-72. In exemplary instances, the liposome comprises 48% cholesterol, 20% DSPC, 2% PEG-c-DMA, and 30% cationic lipid, where the cationic lipid can be 1,2-distearloxy-N, N-dimethylaminopropane (DSDMA), DODMA, DLin-DMA, or 1,2-dilinolenyloxy-3-dimethylaminopropane (DLenDMA), as described by Heyes et al., J. Control Release, 107(2): 276-87 (2005).

In some embodiments, the liposomes comprise from about 25.0% cholesterol to about 40.0% cholesterol, from about 30.0% cholesterol to about 45.0% cholesterol, from about 35.0% cholesterol to about 50.0% cholesterol and/or from about 48.5% cholesterol to about 60% cholesterol. In some embodiments, the liposomes may comprise a percentage of cholesterol selected from the group consisting of 28.5%, 31.5%, 33.5%, 36.5%, 37.0%, 38.5%, 39.0% and 43.5%. In some embodiments, the liposomes may comprise from about 5.0% to about 10.0% DSPC and/or from about 7.0% to about 15.0% DSPC.

In some embodiments, the liposomes are DiLa2 liposomes (Marina Biotech, Bothell, Wash.), SMARTICLES® (Marina Biotech, Bothell, Wash.), neutral DOPC (1,2-dioleoyl-sn-glycero-3-phosphocholine) based liposomes (e.g., siRNA delivery for ovarian cancer (Landen et al. Cancer Biology & Therapy 2006 5(12) 1708-1713); herein incorporated by reference in its entirety) and hyaluronan-coated liposomes (Quiet Therapeutics, Israel).

In various instances, the cationic lipid comprises 2,2-dilinoleyl-4-dimethylaminoethyl-[1,3]-dioxolane (DLin-KC2-DMA), dilinoleyl-methyl-4-dimethylaminobutyrate (DLin-MC3-DMA), or di((Z)-non-2-en-1-yl) 9-((4-(dimethylamino)butanoyl)oxy)heptadecanedioate (L319), and further comprise a neutral lipid, a sterol and a molecule capable of reducing particle aggregation, for example, a PEG or PEG-modified lipid.

The liposome in various aspects comprises DLin-DMA, DLin-K-DMA, 98N12-5, C12-200, DLin-MC3-DMA, DLin-KC2-DMA, DODMA, PLGA, PEG, PEG-DMG, PEGylated lipids and amino alcohol lipids. In some aspects, the liposome comprises a cationic lipid such as, but not limited to, DLin-DMA, DLin-D-DMA, DLin-MC3-DMA, DLin-KC2-DMA, DODMA and amino alcohol lipids. The amino alcohol cationic lipid comprises in some aspects lipids described in and/or made by the methods described in U.S. Patent Publication No. US20130150625, herein incorporated by reference in its entirety. As a non-limiting example, the cationic lipid in certain aspects is 2-amino-3-[(9Z, 12Z)-octadeca-9, 12-dien-1-yloxy]-2-{[(9Z,2Z)-octadeca-9, 12-dien-1-yloxy]methyl}propan-1-ol (Compound 1 in US20130150625); 2-amino-3-[(9Z)-octadec-9-en-1-yloxy]-2-{[(9Z)-octadec-9-en-1-yloxy]methyl}propan-1-ol (Compound 2 in US20130150625); 2-amino-3-[(9Z, 12Z)-octadeca-9, 12-dien-1-yloxy]-2-[(octyloxy)methyl]propan-1-ol (Compound 3 in US20130150625); and 2-(dimethylamino)-3-[(9Z, 12Z)-octadeca-9, 12-dien-1-yloxy]-2-{[(9Z, 12Z)-octadeca-9, 12-dien-1-yloxy]methyl}propan-1-ol (Compound 4 in US20130150625); or any pharmaceutically acceptable salt or stereoisomer thereof.

In various embodiments, the liposome comprises (i) at least one lipid selected from the group consisting of 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319); (ii) a neutral lipid selected from DSPC, DPPC, POPC, DOPE and SM; (iii) a sterol, e.g., cholesterol; and (iv) a PEG-lipid, e.g., PEG-DMG or PEG-cDMA, in a molar ratio of about 20-60% cationic lipid: 5-25% neutral lipid: 25-55% sterol; 0.5-15% PEG-lipid.

In some embodiments, the liposome comprises from about 25% to about 75% on a molar basis of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), e.g., from about 35 to about 65%, from about 45 to about 65%, about 60%, about 57.5%, about 50% or about 40% on a molar basis.

In some embodiments, the liposome comprises from about 0.5% to about 15% on a molar basis of the neutral lipid e.g., from about 3 to about 12%, from about 5 to about 10% or about 15%, about 10%, or about 7.5% on a molar basis. Examples of neutral lipids include, but are not limited to, DSPC, POPC, DPPC, DOPE and SM. In various aspects, the nanoparticle does not comprise a neutral lipid. In some embodiments, the formulation includes from about 5% to about 50% on a molar basis of the sterol (e.g., about 15 to about 45%, about 20 to about 40%, about 40%, about 38.5%, about 35%, or about 31% on a molar basis. An exemplary sterol is cholesterol. In some embodiments, the formulation includes from about 0.5% to about 20% on a molar basis of the PEG or PEG-modified lipid (e.g., about 0.5 to about 10%, about 0.5 to about 5%, about 1.5%, about 0.5%, about 1.5%, about 3.5%, or about 5% on a molar basis). In some embodiments, the PEG or PEG modified lipid comprises a PEG molecule of an average molecular weight of 2,000 Da. In other embodiments, the PEG or PEG modified lipid comprises a PEG molecule of an average molecular weight of less than 2,000, for example around 1,500 Da, around 1,000 Da, or around 500 Da. Examples of PEG-modified lipids include, but are not limited to, PEG-distearoyl glycerol (PEG-DMG) (also referred herein as PEG-C14 or C14-PEG), PEG-CDMA (further discussed in Reyes et al. J. Controlled Release, 107, 276-287 (2005) the contents of which is herein incorporated by reference in its entirety).

In exemplary aspects, the cationic lipid may be selected from (20Z,23Z)-N,N-dimethylnonacosa-20,23-dien-10-amine, (17Z,20Z)-N,N-dimemylhexacosa-17,20-dien-9-amine, (1Z, 19Z)-N,N-dimethylpentacosa-1 6, 19-dien-8-amine, (13Z, 16Z)-N,N-dimethyldocosa-13, 16-dien-5-amine, (12Z, 15Z)-N,N-dimethylhenicosa-12, 15-dien-4-amine, (14Z,17Z)-N,N-dimethyltricosa-14, 17-dien-6-amine, (15Z, 18Z)-N,N-dimethyltetracosa-15, 18-dien-7-amine, (18Z,21Z)-N,N-dimethylheptacosa-18,21-dien-10-amine, (15Z, 18Z)-N,N-dimethyltetracosa-15, 18-dien-5-amine, (14Z, 17Z)-N, N-dimethyltricosa-14, 17-dien-4-amine, (19Z,22Z)-N,N-dimeihyloctacosa-19,22-dien-9-amine, (18Z,21 Z)-N,N-dimethylheptacosa-18,21-dien-8-amine, (17Z,20Z)-N,N-dimethylhexacosa-17,20-dien-7-amine, (16Z,19Z)-N,N-dimethylpentacosa-16, 19-dien-6-amine, (22Z,25Z)-N,N-dimethylhentriaconta-22,25-dien-10-amine, (21 Z,24Z)-N,N-dimethyltriaconta-21,24-dien-9-amine, (18Z)-N,N-dimetylheptacos-18-en-10-amine, (17Z)-N,N-dimethylhexacos-17-en-9-amine, (19Z,22Z)-N,N-dimethyloctacosa-19,22-dien-7-amine, N,N-dimethylheptacosan-10-amine, (20Z,23Z)-N-ethyl-N-methylnonacosa-20,23-dien-10-amine, 1-[(11Z, 14Z)-1-nonylicosa-11, 14-dien-1-yl]pyrrolidine, (20Z)-N,N-dimethylheptacos-20-en-10-amine, (15Z)-N,N-dimethyl eptacos-15-en-10-amine, (14Z)-N,N-dimethylnonacos-14-en-10-amine, (17Z)-N,N-dimethylnonacos-17-en-10-amine, (24Z)-N,N-dimethyltritriacont-24-en-10-amine, (20Z)-N,N-dimethylnonacos-20-en-10-amine, (22Z)-N,N-dimethylhentriacont-22-en-10-amine, (16Z)-N,N-dimethylpentacos-16-en-8-amine, (12Z, 15Z)-N,N-dimethyl-2-nonylhenicosa-12,15-dien-1-amine, (13Z, 16Z)-N,N-dimethyl-3-nonyldocosa-13, 16-dien-1-amine, N, N-dimethyl-1-[(1S,2R)-2-octylcyclopropyl]eptadecan-8-amine, 1-[(1S,2R)-2-hexylcyclopropyl]-N, N-dimethylnonadecan-10-amine, N, N-dimethyl-1-[(1S,2R)-2-octylcyclopropyl]nonadecan-10-amine, N,N-dimethyl-21-[(1S,2R)-2-octylcyclopropyl]henicosan-10-amine, N, N-dimethyl-1-[(1S,2S)-2-{[(1R,2R)-2-pentylcyclopropyl]methyl}cyclopropyl]nonadecan-10-amine, N, N-dimethyl-1-[(1S,2R)-2-octylcyclopropyl]hexadecan-8-amine, N, N-dimethyl-[(1R,2S)-2-undecylcyclopropyl]tetradecan-5-amine, N,N-dimethyl-3-{7-[(1S,2R)-2-octylcyclopropyl]heptyl}dodecan-1-amine, 1-[(1R,2S)-2-heptylcyclopropyl]-N, N-dimethyloctadecan-9-amine, 1-[(1S,2R)-2-decylcyclopropyl]-N,N-dimethylpentadecan-6-amine, N, N-dimethyl-1-[(1S,2R)-2-octylcyclopropyl]pentadecan-8-amine, R-N, N-dimethyl-1-[(9Z, 12Z)-octadeca-9, 12-dien-1-yloxy]-3-(octyloxy)propan-2-amine, S-N,N-dimethyl-1-[(9Z, 12Z)-octadeca-9,12-dien-1-yloxy]-3-(octyloxy)propan-2-amine, 1-{2-[(9Z, 12Z)-octadeca-9, 12-dien-1-yloxy]-1-[(octyloxy)methyl]ethyl}pyrrolidine, (2S)-N, N-dimethyl-1-[(9Z, 12Z)-octadeca-9, 12-dien-1-yloxy]-3-[(5Z)-oct-5-en-1-yloxy]propan-2-amine, 1-{2-[(9Z, 12Z)-octadeca-9, 12-dien-1-yloxy]-1-[(octyloxy)methyl]ethyl}azetidine, (2S)-1-(hexyloxy)-N,N-dimethyl-3-[(9Z, 12Z)-octadeca-9, 12-dien-1-yloxy]propan-2-amine, (2S)-1-(heptyloxy)-N,N-dimethyl-3-[(9Z, 12Z)-octadeca-9, 12-dien-1-yloxy]propan-2-amine, N,N-dimethyl-1-(nonyloxy)-3-[(9Z, 12Z)-octadeca-9, 12-dien-1-yloxy]propan-2-amine, N, N-dimethyl-1-[(9Z)-octadec-9-en-1-yloxy]-3-(octyloxy)propan-2-amine; (2S)-N,N-dimethyl-1-[(6Z,9Z, 12Z)-octadeca-6,9, 12-trien-1-yloxy]-3-(octyloxy)propan-2-amine, (2S)-1-[(11Z, 14Z)-icosa-11, 14-dien-1-yloxy]-N,N-dimethyl-3-(pentyloxy)propan-2-amine, (2S)-1-(hexyloxy)-3-[(11Z, 14Z)-icosa-11,14-dien-1-yloxy]-N,N-dimethylpropan-2-amine, 1-[(11Z,14Z)-icosa-11, 14-dien-1-yloxy]-N, N-dimethyl-3-(octyloxy)propan-2-amine, 1-[(13Z, 16Z)-docosa-13, 16-dien-1-yloxy]-N,N-dimethyl-3-(octyloxy)propan-2-amine, (2S)-1-[(13Z, 16Z)-docosa-13, 16-dien-1-yloxy]-3-(hexyloxy)-N,N-dimethylpropan-2-amine, (2S)-1-[(13Z)-docos-13-en-1-yloxy]-3-(hexyloxy)-N,N-dimethylpropan-2-amine, 1-[(13Z)-docos-13-en-1-yloxy]-N,N-dimethyl-3-(octyloxy)propan-2-amine, 1-[(9Z)-hexadec-9-en-1-yloxy]-N, N-dimethyl-3-(octyloxy)propan-2-amine, (2R)-N,N-dimethyl-H(1-methyloctyl)oxyl-3-[(9Z, 12Z)-octadeca-9,12-dien-1-yloxy]propan-2-amine, (2R)-1-[(3,7-dimethyloctyl)oxy]-N, N-dimethyl-3-[(9Z, 12Z)-octadeca-9, 12-dien-1-yloxy]propan-2-amine, N,N-dimethyl-1-(octyloxy)-3-({8-[(1S,2S)-2-{[(1R,2R)-2-pentylcyclopropyl]methyl}cyclopropyl]octyl}oxy)propan-2-amine, N, N-dimethyl-1-{[8-(2-octylcyclopropyl)octyl]oxy}-3-(octyloxy)propan-2-amine and (11E,20Z,23Z)-N,N-dimethylnonacosa-11,20,2-trien-10-amine or a pharmaceutically acceptable salt or stereoisomer thereof.

In some embodiments, the nanoparticle comprises a lipid-polycation complex. The formation of the lipid-polycation complex may be accomplished by methods known in the art and/or as described in U.S. Patent Publication No. 20120178702, herein incorporated by reference in its entirety. As a non-limiting example, the polycation may include a cationic peptide or a polypeptide such as, but not limited to, polylysine, polyornithine and/or polyarginine. In some embodiments, the composition may comprise a lipid-polycation complex, which may further include a non-cationic lipid such as, but not limited to, cholesterol or dioleoyl phosphatidylethanolamine (DOPE).

In various aspects, the cationic liposomes optionally do not comprise a non-cationic lipid. Neutral molecules, in some aspects, may interfere with coiling/condensation of multi-lamellar nanoparticles resulting in RNA loaded liposomes greater than 200 nm in size. Cationic liposomes generated without helper molecules can comprise a size of about 70-200 nm (or less). These constructs consist essentially of a cationic lipid with negatively charged nucleic acid, and may be formulated in a sealed rotary vacuum evaporator which prevents oxidation of the particles (when exposed to the ambient environment). In this aspect, the absence of a helper lipid optimizes mRNA coiling into tightly packaged multilamellar NPs where each NP contains a greater amount of nucleic acid per particle. Due to increased nucleic acid payload per particle, these multi-lamellar RNA nanoparticles drive significantly greater innate immune responses, which are a significant predictor of efficacy for modulating the immune system.

In some aspects, the nucleic acid molecules are present at a nucleic acid molecule:cationic lipid ratio of about 1 to about 5 to about 1 to about 25. In some aspects, the nucleic acid molecules are present at a nucleic acid molecule:cationic lipid ratio of about 1 to about 5 to about 1 to about 20, optionally, about 1 to about 15, about 1 to about 10, or about 1 to about 7.5. As used herein, the term “nucleic acid molecule:cationic lipid ratio” is meant a mass ratio, where the mass of the nucleic acid molecule is relative to the mass of the cationic lipid. Also, in exemplary aspects, the term “nucleic acid molecule:cationic lipid ratio” is meant the ratio of the mass of the nucleic acid molecule, e.g., RNA, added to the liposomes comprising cationic lipids during the process of manufacturing the ML RNA NPs of the present disclosure. In exemplary aspects, the nanoparticle comprises less than or about 10 μg RNA molecules per 150 μg lipid mixture. In exemplary aspects, the nanoparticle is made by incubating about 10 μg RNA with about 150 μg liposomes. In alternative aspects, the nanoparticle comprises more RNA molecules per mass of lipid mixture. For example, the nanoparticle may comprise more than 10 μg RNA molecules per 150 μg liposomes. The nanoparticle in some instances comprises more than 15 μg RNA molecules per 150 μg liposomes or lipid mixture.

In various aspects, the nucleic acid molecules are RNA molecules, e.g., transfer RNA (tRNA), ribosomal RNA (rRNA), messenger RNA (mRNA). In various aspects, the RNA molecules comprise tRNA, rRNA, mRNA, or a combination thereof. In various aspects, the RNA is total RNA isolated from a cell. In exemplary aspects, the RNA is total RNA isolated from a diseased cell, such as, for example, a tumor cell or a cancer cell. Methods of obtaining total tumor RNA is known in the art and described herein at Example 1.

In the context of a method for treating a solid tumor in a subject comprising a surface antigen negative solid tumor, the first composition comprises a nanoparticle comprising a nucleic acid that encodes the surface antigen (also referred to herein as surface tumor antigen) recognized by the CAR T cell. A number of suitable cancer antigen targets for CAR T cell therapy are known and described in e.g., U.S. Pat. No. 10,688,166 (incorporated by reference in its entirety, and particularly with respect to the disclosure of tumor antigens). The antigen may be, for example, a claudin, CD19, CD20, CD22, CD33, CD166, CD70, CD123, CEA, c-Met, PSMA, GD2, GD3, FRα, CAIX, CD171, EGFRVIII, HER2, mesothelin, CD133, CEACAM5, EGFR, GPC3, PSMA, ROR1, VEGFR2, B7-H3, IL-13Rα, PD-L1, IL-11Rα, EphA2, MAGE, MCAM, NKG2D ligands, TEM1, FAP, GAGE, MUC1, or NY-ESO-1. In various aspects, the surface antigen is CD70 (i.e., the nucleic acid of the nanoparticle of the first composition encodes CD70, and the CAR T cell targets CD70-expressing tumor cells). The sequence of human CD70 is known in the art. See, e.g., UniProtKB No. P32970.

In exemplary instances, the RNA molecules are mRNA. In various aspects of the disclosure, a nanoparticle is used which comprises total RNA isolated from a cell. In various aspects, mRNA is in vitro transcribed mRNA. In various instances, the mRNA molecules are produced by in vitro transcription (IVT). Suitable techniques of carrying out IVT are known in the art. In exemplary aspects, an IVT kit is employed. In exemplary aspects, the kit comprises one or more IVT reaction reagents. As used herein, the term “in vitro transcription (IVT) reaction reagent” refers to any molecule, compound, factor, or salt, which functions in an IVT reaction. For example, the kit may comprise prokaryotic phage RNA polymerase and promoter (T7, T3, or SP6) with eukaryotic or prokaryotic extracts to synthesize proteins from exogenous DNA templates. Optionally, the RNA is in vitro transcribed mRNA, wherein the in vitro transcription template is cDNA made from RNA extracted from a tumor cell. In various aspects, the nanoparticle comprises a mixture of RNA which is RNA isolated from a tumor of a human. Optionally, the tumor is osteosarcoma or a malignant brain tumor, such as, a glioblastoma, medulloblastoma, diffuse intrinsic pontine glioma, or a peripheral tumor with metastatic infiltration into the central nervous system. In various aspects, the RNA comprises a sequence encoding a poly(A) tail so that the in vitro transcribed RNA molecule comprises a poly(A) tail at the 3′ end. In various aspects, the method of making a nanoparticle comprises additional processing steps, such as, for example, capping the in vitro transcribed RNA molecules.

The RNA (e.g., mRNAs) in exemplary aspects encodes a protein. Optionally, the protein is selected from the group consisting of a tumor antigen, a cytokine, and a co-stimulatory molecule. Indeed, the protein is, in some aspects, selected from the group consisting of a tumor antigen, a co-stimulatory molecule, a cytokine, a growth factor, a hematopoietic factor, or a lymphokine, including, e.g., cytokines and growth factors that are effective in inhibiting tumor metastasis, and cytokines or growth factors that have been shown to have an antiproliferative effect on at least one cell population. Such cytokines, lymphokines, growth factors, or other hematopoietic factors include, but are not limited to: M-CSF, GM-CSF, TNF, IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IFN, TNFα, TNF1, TNF2, G-CSF, Meg-CSF, GM-CSF, thrombopoietin, stem cell factor, and erythropoietin. Additional growth factors for use herein include angiogenin, bone morphogenic protein-1, bone morphogenic protein-2, bone morphogenic protein-3, bone morphogenic protein-4, bone morphogenic protein-5, bone morphogenic protein-6, bone morphogenic protein-7, bone morphogenic protein-8, bone morphogenic protein-9, bone morphogenic protein-10, bone morphogenic protein-11, bone morphogenic protein-12, bone morphogenic protein-13, bone morphogenic protein-14, bone morphogenic protein-15, bone morphogenic protein receptor IA, bone morphogenic protein receptor IB, brain derived neurotrophic factor, ciliary neutrophic factor, ciliary neutrophic factor receptor α, cytokine-induced neutrophil chemotactic factor 1, cytokine-induced neutrophil, chemotactic factor 2 α, cytokine-induced neutrophil chemotactic factor 2 β, β endothelial cell growth factor, endothelin 1, epithelial-derived neutrophil attractant, glial cell line-derived neutrophic factor receptor α 1, glial cell line-derived neutrophic factor receptor α 2, growth related protein, growth related protein α, growth related protein β, growth related protein γ, heparin binding epidermal growth factor, hepatocyte growth factor, hepatocyte growth factor receptor, insulin-like growth factor I, insulin-like growth factor receptor, insulin-like growth factor II, insulin-like growth factor binding protein, keratinocyte growth factor, leukemia inhibitory factor, leukemia inhibitory factor receptor α, nerve growth factor nerve growth factor receptor, neurotrophin-3, neurotrophin-4, pre-B cell growth stimulating factor, stem cell factor, stem cell factor receptor, transforming growth factor α, transforming growth factor β, transforming growth factor β1, transforming growth factor β1.2, transforming growth factor β2, transforming growth factor β3, transforming growth factor β5, latent transforming growth factor β1, transforming growth factor β binding protein I, transforming growth factor β binding protein II, transforming growth factor β binding protein III, tumor necrosis factor receptor type I, tumor necrosis factor receptor type II, urokinase-type plasminogen activator receptor, and chimeric proteins and biologically or immunologically active fragments thereof. In exemplary aspects, the tumor antigen is an antigen derived from a viral protein, an antigen derived from point mutations, or an antigen encoded by a cancer-germline gene. In exemplary aspects, the tumor antigen is pp65, p53, KRAS, NRAS, MAGEA, MAGEB, MAGEC, BAGE, GAGE, LAGE/NY-ESO1, SSX, tyrosinase, gp100/pmel17, Melan-A/MART-1, gp75/TRP1, TRP2, CEA, RAGE-1, HER2/NEU, or WT1, or any other tumor antigens described herein. In exemplary aspects, the co-stimulatory molecule is selected from the group consisting of CD80 and CD86.

In some aspects, the methods of the disclosure may comprise use of a nanoparticle comprising nucleic acid that is not mRNA isolated from a tumor (i.e., not tumor mRNA), or is not a nucleic acid that encodes a protein expressed by a tumor cell or by a human (i.e., the protein is not related to a tumor antigen or cancer antigen). In this regard, in various aspects, the nanoparticle does not comprise nucleic acid that encodes a tumor antigen recognized by the CAR T cell to be administered to the subject. In various aspects, the nanoparticle comprises a mixture of nucleic acid wherein only a small percentage encodes a tumor antigen recognized by the CAR T cell to be administered to the subject (e.g., less than 10% or less than 5% of the nucleic acid encodes a tumor antigen recognized by the CAR T cell). In some aspects, the nucleic acid encodes a protein which is non-specific relative to a tumor or cancer. For example, the non-specific protein may be green fluorescence protein (GFP) or ovalbumin (OVA). Surprisingly, nucleic acids encoding the tumor antigen recognized by the CAR T cell are not required to achieve preconditioning using the NP composition of the present disclosure.

In aspects of the disclosure comprising treating a solid tumor in a subject which has a surface antigen negative tumor, the first composition (comprising nanoparticles) may comprise a mixed population of nanoparticles, those which comprise nucleic acid that encode surface antigen and others that that do not comprise nucleic acid that encodes a tumor antigen recognized by the CAR T cell to be administered to the subject.

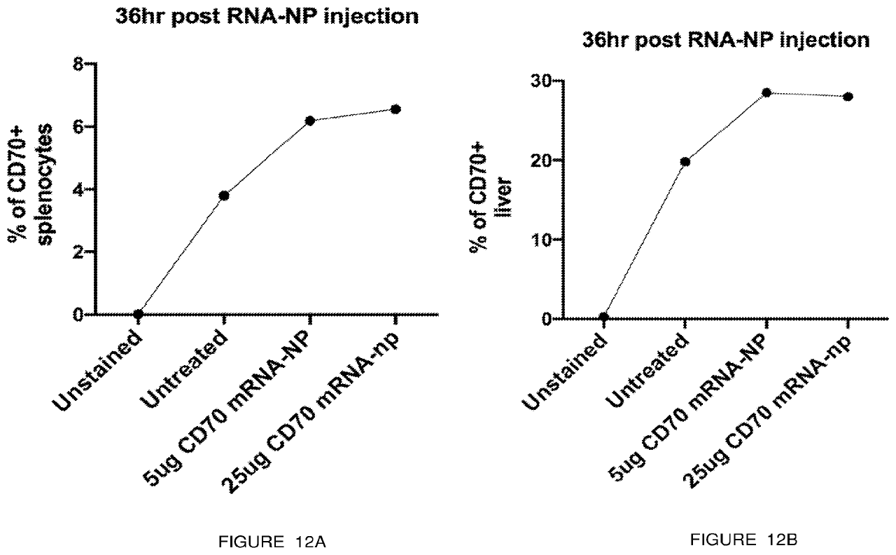

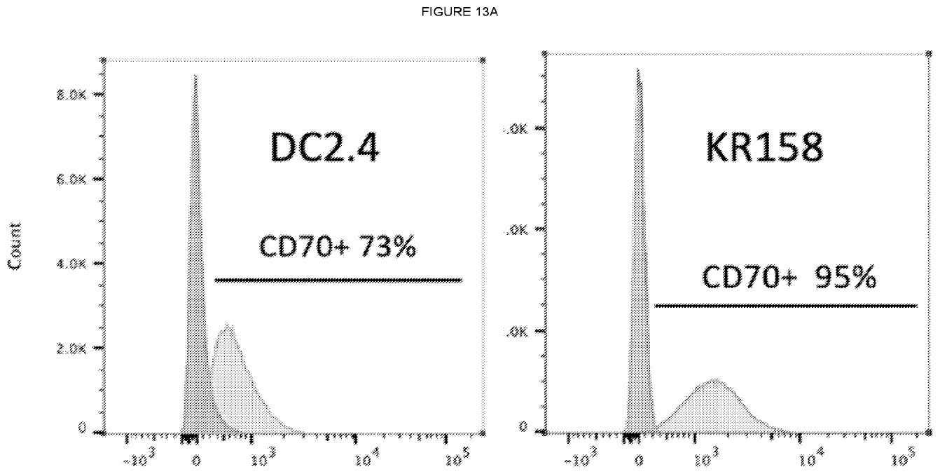

In various aspects, the disclosure contemplates use of nanoparticles wherein the nucleic acid layers comprise a sequence of a nucleic acid molecule expressed by slow-cycling cells (SCCs). The term “slow-cycling cells” or “SCCs” refers to tumor or cancer cells that proliferate at a slow rate. In exemplary aspects, the SCCs have a doubling time of at least about 50 hours. SCCs have been identified in numerous cancer tissues, including, melanoma, ovarian cancer, pancreatic adenocarcinoma, breast cancer, glioblastoma, and colon cancer. As taught in Deleyrolle et al., Brain 134(5): 1331-1343 (2011) (incorporated by reference herein, particularly with respect to the description of SCCs), SCCs display increased tumor-initiation properties and are stem cell like. Because of their slow proliferation rate, SCCs are also referred to as label-retaining cells (LRCs). In exemplary instances, the nucleic acid molecules are RNA extracted from isolated SCCs or are nucleic acid molecules which hybridize to RNA extracted from isolated SCCs. Optionally, the SCCs are isolated from a mixed tumor cell population obtained from a subject with a tumor (e.g., a glioblastoma). As used herein, the term “mixed tumor cell population” refers to a heterogeneous cell population comprising tumor cells of different sub-types and comprising slow-cycling cells and at least one other tumor cell type, e.g., fast-cycling cells (FCCs). NP comprising nucleic acid layers comprising a sequence of a nucleic acid molecule expressed by slow-cycling cells (SCCs) are further described in International Patent Application No. PCT/US21/16925 (WO 2021/158996), which is hereby incorporated by reference in its entirety, particularly with respect to FIG. 12.

Optionally, various aspects of the disclosure may involve use of a nanoparticle comprising RNA molecules that bind to or encode an epitope of a nucleic acid encoding a fusion protein expressed by a tumor. In exemplary aspects, the epitope comprises a junction of the nucleic acid encoding the fusion protein. In various aspects, the epitope encodes an amino acid sequence which binds to an MHC Class II. By way of example, the fusion protein in various instances is a C11orf95-RELA fusion protein or a fusion protein described herein or in Parker and Zhang, Chin J Cancer 32(11): 594-603 (2013); Ding et al., In J Mol Sci 19(1): 177 (2018); Wener et al., Molecular Cancer 17, article number 28 (2018); or Yu et al., Scientific Reports 9, article number 1074 (2019). See FIGS. 25-28 of International Patent Application No. PCT/US21/16925, hereby incorporated by reference. In exemplary aspects, the fusion protein is a fusion protein that comprises at least a portion of two of Erdr1, Mid1, Ppp1r13b, or CKB. In exemplary aspects, the fusion protein is a fusion protein comprising at least a portion of Erdr1 and at least a portion of Mid1 (e.g., Erdr1/Mid1 or Mid1/Erdr1) or at least a portion of Ppp1r13b and at least a portion of CKB (e.g., Ppp1r13b/CKB or CKB/Ppp1r13b). In various aspects, the fusion protein is expressed by a murine model of a brain tumor. In exemplary aspects, the fusion protein is a fusion protein comprising at least a portion of two of EWSR1, FUSR1, FOXO1, SS18, FLI1, ERG, ETV1, ETV4, FEV, SSX1. In exemplary aspects, the fusion protein comprises at least a portion of EWSR1, FUSR1, FOXO1, or SS18 and at least a portion of FLI1, ERG, ETV1, ETV4, FEV, or SSX1 (e.g., EWSR1/FLI1, FLI1/EWSR1, EWSR1/ERG, ERG/EWSR1, EWSR1/ETV1 or ETV1/EWSR1, EWSR1/ETV4, ETV4/EWSR1, EWSR1/FEV, FEV/EWSR1, FUSR1/FEV, FEV/FUSR1, FUSR1/ERG, ERG/FUSR1, FOXO1/PAX3, PAX3/FOXO1, FOXO1/PAX7, PAX7/FOXO1, SS18/SSX1, or SSX1/SS18). In exemplary aspects, the fusion protein is expressed by a sarcoma tumor. In various aspects, the fusion protein comprises at least a portion of two of YAP1, FAM118B, MAMLD1, C11or95, RELA, EPN, MTOR, CASZ1, TP53, DEK, FXR2, BRAF, KIAA1549, or EML4. In exemplary aspects, the fusion protein comprises at least a portion of YAP1, C11or95, MTOR, TP53 or BRAF and at least a portion of FAM118B, MAMLD1, RELA, C11orf95, EPN, CASZ1, DEK, FXR2, KIAA1549, or EML4 (e.g., YAP1/FAM118B, FAM118B/YAP1, YAP1/MAMLD1, MAMLD1/YAP1, YAP1/C11orf95, c11orf95/YAP1, C11orf95-RELA, c11orf95/RELA, EPN-YAP1, EPN-YAP1, MTOR/MTOR, MTOR/CASZ1, CASZ1/MTOR, TP53/TP53, TP53/DEK, DEK/TP53, TP53/FXR2, FXR2/TP53, BRAF/KIAA1549, KIAA1549/BRAF, BRAF/EML4, or EML4/BRAF). In exemplary aspects, the fusion protein is expressed by a neuro-tumor. The fusion protein may be any one of those described at the website for the Catalog of Somatic Mutations in Cancer (COSMIC) at cancer.sanger.ac.uk/cosmic/fusion or at the website for the Atlas of Genetics and Cytogenetics in Oncology and Haematology at atlasgeneticsoncology.org/Deep/Cancer_CytogenomicsID20145.html. Nanoparticles comprising RNA molecules bind to or encode an epitope of a nucleic acid encoding a fusion protein expressed by a tumor are further described in International Patent Application No. PCT/US21/16925, which is hereby incorporated by reference in its entirety.

In various instances, aspects of the disclosure may involve use of nanoparticles comprising RNA molecules that are antisense molecules, optionally siRNA, shRNA, miRNA (microRNA), or any combination thereof. The antisense molecule can be one which mediates RNA interference (RNAi). As known by one of ordinary skill in the art, RNAi is a ubiquitous mechanism of gene regulation in plants and animals in which target mRNAs are degraded in a sequence-specific manner (Sharp, Genes Dev., 15, 485-490 (2001); Hutvagner et al., Curr. Opin. Genet. Dev., 12, 225-232 (2002); Fire et al., Nature, 391, 806-811 (1998); Zamore et al., Cell, 101, 25-33 (2000)). The natural RNA degradation process is initiated by the dsRNA-specific endonuclease Dicer, which promotes cleavage of long dsRNA precursors into double-stranded fragments between 21 and 25 nucleotides long, termed small interfering RNA (siRNA; also known as short interfering RNA) (Zamore et al., Cell, 101, 25-33 (2000); Elbashir et al., Genes Dev., 15, 188-200 (2001); Hammond et al., Nature, 404, 293-296 (2000); Bernstein et al., Nature, 409, 363-366 (2001)). siRNAs are incorporated into a large protein complex that recognizes and cleaves target mRNAs (Nykanen et al., Cell, 107, 309-321 (2001)). It has been reported that introduction of dsRNA into mammalian cells does not result in efficient Dicer-mediated generation of siRNA and therefore does not induce RNAi (Caplen et al., Gene, 252, 95-105 (2000); Ui-Tei et al., FEBS Lett, 479, 79-82 (2000)). The requirement for Dicer in maturation of siRNAs in cells can be bypassed by introducing synthetic 21-nucleotide siRNA duplexes, which inhibit expression of transfected and endogenous genes in a variety of mammalian cells (Elbashir et al., Nature, 411: 494-498 (2001)). Aspects of the disclosure may involve use of nanoparticles comprising RNA molecules that mediate RNAi, and in some aspects the RNA is a siRNA molecule specific for inhibiting the expression of a protein. The term “siRNA” as used herein refers to an RNA (or RNA analog) comprising from about 10 to about 50 nucleotides (or nucleotide analogs) which is capable of directing or mediating RNAi. In exemplary embodiments, an siRNA molecule comprises about 15 to about 30 nucleotides (or nucleotide analogs) or about 20 to about 25 nucleotides (or nucleotide analogs), e.g., 21-23 nucleotides (or nucleotide analogs). The siRNA can be double or single stranded, preferably double-stranded.

In alternative aspects, the disclosure contemplates use of a nanoparticle comprising RNA molecules that are short hairpin RNA (shRNA) molecules specific for inhibiting the expression of a protein. The term “shRNA” as used herein refers to a molecule of about 20 or more base pairs in which a single-stranded RNA partially contains a palindromic base sequence and forms a double-strand structure therein (i.e., a hairpin structure). An shRNA can be an siRNA (or siRNA analog) which is folded into a hairpin structure. shRNAs typically comprise about 45 to about 60 nucleotides, including the approximately 21 nucleotide antisense and sense portions of the hairpin, optional overhangs on the non-loop side of about 2 to about 6 nucleotides long, and the loop portion that can be, e.g., about 3 to 10 nucleotides long.

In exemplary aspects, disclosure contemplates use of nanoparticles comprising an antisense molecule which is a microRNA (miRNA). As used herein the term “microRNA” refers to a small (e.g., 15-22 nucleotides), non-coding RNA molecule which base pairs with mRNA molecules to silence gene expression via translational repression or target degradation. microRNA and the therapeutic potential thereof are described in the art. See, e.g., Mulligan, MicroRNA: Expression, Detection, and Therapeutic Strategies, Nova Science Publishers, Inc., Hauppauge, N Y, 2011; Bader and Lammers, “The Therapeutic Potential of microRNAs” Innovations in Pharmaceutical Technology, pages 52-55 (March 2011).

In certain instances, the RNA molecule is an antisense molecule, optionally, an siRNA, shRNA, or miRNA, which targets a protein of an immune checkpoint pathway for reduced expression. In various aspects, the protein of the immune checkpoint pathway is CTLA-4, PD-1, PD-L1, PD-L2, B7-H3, B7-H4, TIGIT, LAG3, CD112 TIM3, BTLA, or co-stimulatory receptor ICOS, OX40, 41BB, or GITR. The protein of the immune-checkpoint pathway in certain instances is CTLA4, PD-1, PD-L1, B7-H3, B7H4, or TIM3. Immune checkpoint signaling pathways are reviewed in Pardoll, Nature Rev Cancer, 12(4): 252-264 (2012).

In exemplary embodiments, the nanoparticles of the present disclosure comprise a mixture of RNA molecules. In exemplary aspects, the mixture of RNA molecules is RNA isolated from cells from a human and optionally, the human has a tumor. In some aspects, the mixture of RNA is RNA isolated from the tumor of the human. In exemplary aspects, the human has cancer, optionally, any cancer described herein. Optionally, the tumor from which RNA is isolated is selected from the group consisting of a glioma (including, but not limited to, a glioblastoma), a medulloblastoma, a diffuse intrinsic pontine glioma, or a peripheral tumor with metastatic infiltration into the central nervous system (e.g., melanoma or breast cancer). Optionally, the tumor from which RNA is isolated is osteosarcoma. In exemplary aspects, the tumor from which RNA is isolated is a tumor of a cancer, e.g., any of these cancers described herein.

In various aspects, the nanoparticles comprise a nucleic acid molecule (e.g., RNA molecule) comprising a nucleotide sequence encoding a chimeric protein comprising a LAMP protein. In certain aspects, the LAMP protein is a LAMP1, LAMP 2, LAMP3, LAMP4, or LAMP5 protein.

### CAR T Cells

The compositions disclosed herein are part of a treatment regimen for subjects undergoing treatment with T cells expressing a chimeric antigen receptor (CAR T cell). Generally, the method described herein is not dependent on a particular CAR T cell product. In various aspects, the CAR T cell binds the surface antigen encoded by nanoparticles of the first composition. In various aspects, the method of the disclosure is not dependent on a particular target cell for which an immune response is desired. “Chimeric antigen receptor” or “CAR” refers to an artificial immune cell receptor that is engineered to recognize and bind to an antigen expressed by a target cell, such as a tumor cell. Generally, a CAR is designed for a T cell and is a chimera of a signaling domain of the T cell receptor (TCR) complex and an antigen-recognizing domain (e.g., a single chain fragment (scFv) of an antibody or other antibody fragment) (Enblad et al., Human Gene Therapy, 26(8):498-505 (2015)). CARs have the ability to redirect T cell specificity and reactivity toward a selected target in a non-MHC-restricted manner. The non-MHC-restricted antigen recognition gives T cells expressing CARs the ability to recognize an antigen independent of antigen processing, thus bypassing a major mechanism of tumor escape. Moreover, when expressed in T cells, CARs advantageously do not dimerize with endogenous T-cell receptor (TCR) alpha and beta chains.

There are various formats of CARs, each of which contains different components. “First generation” CARs join an antibody-derived scFv to the CD3zeta (ζ or z) intracellular signaling domain of the T-cell receptor through hinge and transmembrane domains. “Second generation” CARs incorporate an additional domain, e.g., CD28, 4-1BB (41BB), or ICOS, to supply a costimulatory signal. “Third generation” CARs contain two costimulatory domains fused with the TCR CD3zeta chain. Third generation costimulatory domains may include, e.g., a combination of CD3zeta, CD27, CD28, 4-1BB, ICOS, or OX40. CARs, in some embodiments, contain an ectodomain (e.g., CD3zeta), commonly derived from a single chain variable fragment (scFv), a hinge, a transmembrane domain, and an endodomain with one (first generation), two (second generation), or three (third generation) signaling domains derived from CD3 and/or co-stimulatory molecules (Maude et al., Blood, 125(26):4017-4023 (2015); Kakarla and Gottschalk, Cancer J., 20(2): 151-155 (2014)).

In various aspects, the CAR T cell targets a tumor antigen. Tumor antigens include, e.g., moieties associated with the cell surface of a cancer cell and is preferably not (or only rarely) expressed in normal (non-cancerous) tissues. A number of suitable cancer antigen targets for CAR T cell therapy are known and described in e.g., U.S. Pat. No. 10,688,166 (incorporated by reference in its entirety, and particularly with respect to the disclosure of tumor antigens). For example, the CAR T cell may target, e.g., a claudin, CD19, CD20, CD22, CD33, CD70, CD123, mesothelin, CEA, c-Met, PSMA, GD-2, or NY-ESO-1 (or any other antigen described herein). Examples of CAR T cell therapeutics for the treatment of cancer include, e.g., BREYANZI® (lisocabtagene maraleucel), TECARTUS™ (brexucabtagene autoleucel), KYMRIAH™ (tisagenlecleucel), and YESCARTA™ (axicabtagene ciloleucel). Examples of non-cancer related antigens include viral antigens (e.g., human immunodeficiency virus (HIV) antigens, hepatitis C virus (HCV) antigens, hepatitis B virus (HBV) antigens, cytomegalovirus (CMV) antigens, Epstein Barr virus (EBV) antigens), fungal antigens, parasitic antigens, and bacterial antigens.

In various aspects, the CAR of the modified T cell comprises an antigen binding domain that binds Cluster of Differentiation 70 (CD70). CD70 is a type II transmembrane protein that represents the only ligand for CD27. CD70 is a glycosylated transmembrane protein of the tumor necrosis factor receptor family. CD70-CD27 interactions play an important role in providing co-stimulation during the development of functional lymphocytes; strict control of CD70 expression is required for optimal signaling for immune cell activation. While CD70 expression is restricted to highly activated T/B lymphocytes and a small subset of mature dendritic cells, distinct solid tumor malignancies, including osteosarcoma, may constitutively overexpress CD70. CD70 is not only highly expressed by primary tumors, but also in recurrent tumors, which presents a consistent therapeutic target for primary and recurrent tumors. In various aspects of the disclosure, the CAR comprises an antigen binding portion which comprises an extracellular part of CD27 (the ligand for CD70). Optionally, the transmembrane domain is an intracellular part of 41BB. An exemplary sequence for a CD70-targeted CAR is encoded by SEQ ID NO:1: TGGCAAG ACCCCACCCC TGGTGGCTGT GTGTGCTGGG CACACTGGTC GGACTGAGCG CCACCCCTGC CCCTAAGAGC TGCCCCGAGA GACACTACTG GGCTCAGGGC AAGCTGTGCT GCCAGATGTG CGAGCCCGGC ACCTTCCTGG TGAAAGACTG CGACCAGCAC CGGAAGGCCG CCCAGTGCGA TCCTTGCATC CCCGGCGTGT CCTTCAGCCC CGACCACCAC ACCAGACCCC ACTGCGAGAG CTGCCGGCAT TGCAACTCTG GCCTGCTGGT CCGCAACTGC ACCATCACCG CCAACGCCGA GTGCGCCTGC AGAAACGGCT GGCAGTGCCG GGACAAAGAA TGCACCGAGT GCGACCCTCT GCCCAACCCC AGCCTGACCG CCAGAAGCAG CCAGGCTCTG AGCCCTCACC CTCAGCCCAC CCATCTGCCC TACGTGTCCG AGATGCTGGA AGCCCGGACA GCCGGCCACA TGCAGACCCT GGCCGACTTC AGACAGCTGC CCGCCAGAAC CCTGAGCACC CACTGGCCTC CCCAGCGGAG CCTGTGCAGC AGCGACTTCA TCCGGATCCT GGTGATCTTC AGCGGCATGT TCCTGGTGTT CACCCTGGCT GGCGCCCTGT TCCTGCACAA GCGGGGCAGA AAGAAGCTGC TGTACATCTT CAAGCAGCCC TTCATGCGGC CCGTGCAGAC CACCCAGGAA GAGGACGGCT GCAGCTGCCG GTTCCCCGAG GAAGAGGAAG GCGGCTGCGA GCTGAGAGTG AAGTTCAGCA GAAGCGCCGA CGCCCCTGCC TACCAGCAGG GCCAGAACCA GCTGTACAAC GAGCTGAACC TGGGCAGACG GGAAGAGTAC GACGTGCTGG ACAAGCGGAG AGGCCGGGAC CCTGAGATGG GCGGCAAGCC CCAGAGGCGG AAGAACCCTC AGGAAGGCCT GTATAACGAA CTGCAGAAAG ACAAGATGGC CGAGGCCTAC AGCGAGATCG GCATGAAGGG CGAGCGGCGG AGAGGCAAGG GCCACGATGG CCTGTACCAG GGCCTGAGCA CCGCCACCAA GGACACCTAC GACGCCCTGC ACATGCAGGC TCTGCCTCCA AGA (SEQ ID NO: 1). Exemplary CD70-targeted CAR T cells are described in International Patent Publication No. WO 2019/051047, incorporated herein by reference in its entirety and in particular with respect to its disclosure of chimeric antigen receptors and production of CAR T cells.

A CAR T therapy may be an immunotherapy utilizing a subject or a patient s own immune cells that are engineered to be able to produce a particular CAR(s) on their surface. In some situations, T cells are collected from the body of a subject or a patient via apheresis, a process that withdraws blood from the body and removes one or more blood components (such as plasma, platelets or white blood cells). The T cells collected from the body are then genetically engineered to produce a particular chimeric antigen receptor on their surface. The CAR T cells are expanded by growing in a laboratory and then administered to the subject or patient, or another subject or patient. The CAR T cells will recognize and kill cancer cells that express a targeted antigen on their surface. The cells may be isolated from the subject which will be recipient of the therapy, or may be isolated from a donor subject that is not ultimate recipient of the therapy.

### Use

The disclosure provides methods for, e.g., enhancing the efficacy of CAR T cell therapy and/or making a tumor susceptible to treatment using CAR T cells. For example, the disclosure provides a method of preconditioning a subject for chimeric antigen receptor (CAR) T cell therapy, the method comprising administering to the subject a first composition comprising a nanoparticle comprising a positively-charged surface and an interior comprising (i) a core and (ii) at least two nucleic acid layers, wherein each nucleic acid layer is positioned between a cationic lipid bilayer, at least one day prior to administering CAR T cell therapy to the subject. In exemplary aspects, the nucleic acid molecules are mRNA. Optionally, the composition is systemically administered to the subject. For example, the composition is administered intravenously. In various aspects, the pharmaceutical composition is administered in an amount which is effective to activate dendritic cells (DCs) in the subject.

The first nanoparticle composition is administered to the subject at least one day prior to administration of CAR T cells (i.e., one or more days prior to administration of the CAR T cells), in various aspects of the disclosure. In an exemplary aspect of the disclosure, the first composition is administered to the subject at least one day but no longer than about 30 days prior to administration of the CAR T cells. For example, the first composition is optionally administered between about two and about 21 days prior to administration of the CAR T cells (e.g., administered between about two and about 14 days prior to administration of the CAR T cells). For example, the method may comprise administering the composition about five to about eight days prior to administering the CAR T cell therapy to the subject, such as about seven days prior to administering the CAR T cell therapy. In various aspects, the first composition is administered between two hours and 72 hours prior to CAR T cell administration. Multiple administrations of the composition may be given to the subject, so long as the administrations are at least one day prior to the CAR T cell administration. Preconditioning with the first nanoparticle composition of the disclosure, e.g., primes the body for receipt of the CAR T cell product, facilitating trafficking to target cells and enhancing persistence and activation of the CAR T cells. In some aspects, the method further comprises the step of administering CAR T cells to the subject.

The disclosure further provides a method of treating a solid tumor in a subject, the method comprising administering to a subject comprising a surface antigen negative solid tumor a first composition comprising a nanoparticle comprising a positively-charged surface and an interior comprising (i) a core and (ii) at least two nucleic acid layers, wherein each nucleic acid layer is positioned between a cationic lipid bilayer and the nucleic acid encodes the surface antigen, and administering a second composition comprising a CAR T cell that targets the surface antigen. In exemplary aspects, the nucleic acid molecules are mRNA. The first nanoparticle composition is optionally administered to the subject at least one day prior to administration of the second composition comprising the CAR T cells (i.e., one or more days prior to administration of the CAR T cells). In various aspects, the first composition is administered to the subject at least one day but no longer than about 30 days prior to administration of the CAR T cells. For example, the first composition is optionally administered at least once between about two and about 21 days prior to administration of the CAR T cells (e.g., administered between about two and about 14 days prior to administration of the CAR T cells). For example, the method may comprise administering the first composition about two to about five days prior to administering the CAR T cell therapy to the subject. Multiple administrations of the first composition may be given to the subject prior to the CAR T cell administration, and multiple administrations of the first composition may be given to the subject after the CAR T cell administration. Optionally, the first and/or second compositions are systemically administered to the subject. For example, the first composition and/or second composition is administered intravenously.

In various aspects of the disclosure, the subject comprises a surface antigen negative tumor. In this regard, a surface antigen negative tumor is a tumor which expresses insufficient levels of surface tumor antigen to achieve a clinically relevant response to CAR T cell therapy prior to the instant method. “Surface antigen negative tumor” does not necessarily require that the surface antigen is completely absent from the tumor, although this is contemplated by the disclosure. A surface antigen negative tumor, in various respects, is a tumor wherein less than 20% of the cells of the tumor (e.g., as measured by tumor biopsy) expresses the surface antigen (e.g., less than 18%, less than 15%, less than 13%, less than 10%, less than 8%, less than 5%, or less than 3% of the cells express the surface antigen). Thus, in various aspects, the surface antigen negative tumor, prior to the instant method, is one in which 19%, 18%, 17%, 16%, 15%, 14%, 13%, 12%, 11%, 10%, 9%, 8%, 7%, 6%, 5%, 4%, 3%, 2%, 1%, or less of the cells in the tumor express the surface antigen. Methods of characterizing the presence or absence of a surface tumor antigen on tumor cells are well known in the art and include, for example, PCR detection methods, next generation sequencing methods, fluorescence-activated cell sorting (FACS), and immunostaining.

Optionally, the subject is not administered lymphodepletion therapy within 21 days prior to administration of the CAR T cell therapy. Lymphodepletion therapy is understood in the art and comprises, for example, administration of chemotherapeutic agents, such as cyclophosphamide, fludarabine, pentostatin, or bendamustine, or irradiation (e.g. total body irradiation). Optionally, the subject is not administered lymphodepletion therapy within 18 days, within 14 days, within 10 days, within 7 days, or within 3 days prior to administration of the CAR T cell therapy. Also optionally, the subject is not administered lymphodepletion therapy within 21 days prior to administration of the first nanoparticle composition. Optionally, the subject is not administered lymphodepletion therapy within 18 days, within 14 days, within 10 days, within 7 days, or within 3 days prior to administration of the first nanoparticle composition.

In various aspects, the method further comprises administering an additional (a second or third) composition comprising a nanoparticle comprising a positively-charged surface and an interior comprising (i) a core and (ii) at least two nucleic acid layers, wherein each nucleic acid layer is positioned between a cationic lipid bilayer, after administration of the CAR T cell therapy. The discussion above with respect to nanoparticles, nucleic acids, etc., apply to both the first composition and the additional composition disclosed herein. The first composition and the additional composition may be the same composition, or they may be different. For example, the nanoparticles of the additional composition may comprise different nucleic acids compared to the nanoparticles of the first composition. In various aspects, the nucleic acid incorporated into the nanoparticles of the additional composition is tumor mRNA, such as mRNA is in vitro transcribed mRNA wherein the in vitro transcription template is cDNA made from RNA extracted from a tumor cell. In various aspects, the additional composition is administered between two hours and 72 hours after CAR T cell administration, although the disclosure contemplates other timing of administration.

The present disclosure also provides a method of increasing the sensitivity of a solid tumor to treatment with CAR T cells. In exemplary embodiments, the method comprises administering to the subject a first composition comprising a nanoparticle described herein, e.g., a nanoparticle comprising a positively-charged surface and an interior comprising (i) a core and (ii) at least two nucleic acid layers, wherein each nucleic acid layer is positioned between a cationic lipid bilayer. Optionally, the nucleic acid encodes a tumor surface antigen. Optionally, the first composition is administered at least one day prior to administering the CAR T cells. In exemplary aspects, “sensitivity” means “responsive to treatment” and the concepts of “sensitivity” and “responsiveness” are positively associated in that a tumor or cancer cell that is responsive to a drug/compound treatment is said to be sensitive to that drug. “Sensitivity” may be measured or described quantitatively in terms of the point of intersection of a dose-effect curve with the axis of abscissal values or a line parallel to it; such a point corresponds to the dose just required to produce a given degree of effect. In analogy to this, the “sensitivity” of a measuring system is defined as the lowest input (smallest dose) required producing a given degree of output (effect). The increase in sensitivity provided by the methods of the present disclosure may be at least or about a 1% to about a 10% increase (e.g., at least or about a 1% increase, at least or about a 2% increase, at least or about a 3% increase, at least or about a 4% increase, at least or about a 5% increase, at least or about a 6% increase, at least or about a 7% increase, at least or about a 8% increase, at least or about a 9% increase, at least or about a 9.5% increase, at least or about a 9.8% increase, at least or about a 10% increase) relative to a control. The increase in sensitivity provided by the methods of the present disclosure may be at least or about a 10% to greater than about a 95% increase (e.g., at least or about a 10% increase, at least or about a 20% increase, at least or about a 30% increase, at least or about a 40% increase, at least or about a 50% increase, at least or about a 60% increase, at least or about a 70% increase, at least or about a 80% increase, at least or about a 90% increase, at least or about a 95% increase, at least or about a 98% increase, at least or about a 100% increase) relative to a control. In exemplary aspects, the control is cancer or tumor or a subject or a population of subjects that was not treated with the presently disclosed pharmaceutical composition or wherein the subject or population of subjects was treated with a placebo. In exemplary aspects, the control is the tumor prior to treatment as disclosed herein.

Increased sensitivity to CAR T cell therapy may be determined in any of a number of ways. For example, the method described herein may enhance T cell survival, promote T cell longevity, and/or restrict loss of replicative potential, as well as shrink the tumor and/or mediate tumor cell death. Methods of measuring T cell activity and immune responses are known in the art. T cell activity can be measured by, for example, a cytotoxicity assay, such as those described in Fu et al., PLOS ONE 5(7): e11867 (2010). Other T cell activity assays are described in Bercovici et al., Clin Diagn Lab Immunol. 7(6): 859-864 (2000). Methods of measuring immune responses are described in e.g., Macatangay et al., Clin Vaccine Immunol, 17(9): 1452-1459 (2010), and Clay et al., Clin Cancer Res., 7(5): 1127-35 (2001).

The materials and methods described herein are useful, e.g., for treating a subject for a disease or disorder, such as cancer (e.g., a solid tumor). As used herein, the term “treat,” as well as words related thereto, does not necessarily imply 100% or complete treatment or remission. Rather, there are varying degrees of treatment of which one of ordinary skill in the art recognizes as having a potential benefit or therapeutic effect. In this respect, the methods of treating a disease or disorder can provide any amount or any level of treatment. Furthermore, the treatment provided by the method may include treatment of one or more conditions or symptoms or signs of the disease being treated. For instance, the treatment method of the present disclosure may inhibit one or more symptoms of the disease. Also, the treatment provided by the methods of the present disclosure may encompass slowing the progression of the disease.

The term “treat” also encompasses prophylactic treatment of the disease. Accordingly, the treatment provided by the presently disclosed method may delay the onset or reoccurrence/relapse of the disease being prophylactically treated. In exemplary aspects, the method delays the onset of the disease by 1 day, 2 days, 4 days, 6 days, 8 days, 10 days, 15 days, 30 days, two months, 4 months, 6 months, 1 year, 2 years, 4 years, or more. The prophylactic treatment encompasses reducing the risk of the disease being treated. In exemplary aspects, the method reduces the risk of the disease 2-fold, 5-fold, 10-fold, 20-fold, 50-fold, 100-fold, or more.

In certain aspects, the method of treating the disease may be regarded as a method of inhibiting the disease, or a symptom thereof. As used herein, the term “inhibit” and words stemming therefrom may not be a 100% or complete inhibition or abrogation. Rather, there are varying degrees of inhibition of which one of ordinary skill in the art recognizes as having a potential benefit or therapeutic effect. The presently disclosed methods may inhibit the onset or re-occurrence of the disease or a symptom thereof to any amount or level. In exemplary embodiments, the inhibition provided by the methods is at least or about a 10% inhibition (e.g., at least or about a 20% inhibition, at least or about a 30% inhibition, at least or about a 40% inhibition, at least or about a 50% inhibition, at least or about a 60% inhibition, at least or about a 70% inhibition, at least or about an 80% inhibition, at least or about a 90% inhibition, at least or about a 95% inhibition, at least or about a 98% inhibition). The materials and methods may inhibit the spread or growth of a tumor in any amount or level.

Treatment for cancer (e.g., a solid tumor) may be determined by any of a number of ways. Any improvement in the subject s wellbeing is contemplated (e.g., at least or about a 10% reduction, at least or about a 20% reduction, at least or about a 30% reduction, at least or about a 40% reduction, at least or about a 50% reduction, at least or about a 60% reduction, at least or about a 70% reduction, at least or about an 80% reduction, at least or about a 90% reduction, or at least or about a 95% reduction of any parameter described herein). For example, a therapeutic response would refer to one or more of the following improvements in the disease: (1) a reduction in the number of neoplastic cells; (2) an increase in neoplastic cell death; (3) inhibition of neoplastic cell survival; (5) inhibition (i.e., slowing to some extent, preferably halting) of tumor growth or appearance of new lesions; (6) decrease in tumor size or burden; (7) absence of clinically detectable disease, (8) decrease in levels of cancer markers; (9) an increased patient survival rate; and/or (10) some relief from one or more symptoms associated with the disease or condition (e.g., pain). For example, the efficacy of treatment may be determined by detecting a change in tumor mass and/or volume after treatment. The size of a tumor may be compared to the initial size and dimensions as measured by CT, PET, mammogram, ultrasound, or palpation, as well as by caliper measurement or pathological examination of the tumor after biopsy or surgical resection. Response may be characterized quantitatively using, e.g., percentage change in tumor volume (e.g., the method of the disclosure results in a reduction of tumor volume by at least 10%, at least 20%, at least 30%, at least 40%, at least 50%, at least 60%, at least 70%, at least 80%, or at least 90%). Alternatively, tumor response or cancer response may be characterized in a qualitative fashion like “pathological complete response” (pCR), “clinical complete remission” (cCR), “clinical partial remission” (cPR), “clinical stable disease” (cSD), “clinical progressive disease” (cPD), or other qualitative criteria. In addition, treatment efficacy also can be characterized in terms of responsiveness to other immunotherapy treatment or chemotherapy. In various aspects, the methods of the disclosure further comprise monitoring treatment in the subject.

With regard to the foregoing methods, the composition comprising the nanoparticles, in some aspects, is systemically administered to the subject. Optionally, the method comprises administration of any of the compositions described herein by way of parenteral administration. Parenteral dosage forms of any agent described herein can be administered to a subject by various routes, including, but not limited to, epidural, intracerebral, intracerebroventricular, epicutaneous, intraarterial, intraarticular, intracardiac, intracavernous injection, intradermal, intralesional, intramuscular, intraocular, intraosseous infusion, intraperitoneal, intrathecal, intrauterine, intravaginal administration, intravenous, intravesical, intravitreal, subcutaneous, transdermal, perivascular administration, or transmucosal. For administration to the brain, a pharmaceutical composition can be introduced into tumor tissue using an intratumoral delivery catheter, ventricular shunt catheter attached to a reservoir (e.g., Omaya reservoir), infusion pump, or introduced into a tumor resection cavity (such as Gliasite, Proxima Therapeutics). Tumor tissue in the brain also can be contacted by administering a pharmaceutical composition via convection using continuous infusion catheter or through cerebrospinal fluid. In various instances, the composition is administered to the subject intravenously.

The amount or dose of an active agent (i.e., the “effective amount”) administered should be sufficient to achieve a desired biological effect, e.g., a therapeutic or prophylactic response, in the subject over a reasonable time frame. For example, one or more doses of the composition should be sufficient to, e.g., prime the subject for CAR T cell therapy and/or sensitize a tumor to CAR T cell therapy (and optionally treat a cancer) in a clinically acceptable period of time from the time of administration. In instances where a first composition comprising a nanoparticle comprising nucleic acid which encodes a surface antigen is administered, one or more doses of the first composition should be sufficient to increase the presence of the surface antigen within the tumor. For example, in exemplary aspects, one or more doses of the first composition are provided to the subject to achieve expression of the surface antigen in 20% or more of the tumor cells in the tumor (e.g., to achieve expression of the surface antigen in at least 20%, at least 25%, at least 30%, at least 35%, at least 40%, at least 45%, or at least 50% of tumor cells within the solid tumor in a clinically acceptable period of time from the time of administration. By way of example and not intending to limit the present disclosure, the dose of the active agents of the present disclosure can be about 0.0001 to about 1 g/kg body weight of the subject being treated/day, from about 0.0001 to about 0.001 g/kg body weight, or about 0.01 mg to about 1 g/kg body weight.

In various aspects, the nanoparticle composition is administered according to any regimen including, for example, daily (1 time per day, 2 times per day, 3 times per day, 4 times per day, 5 times per day, 6 times per day), three times a week, twice a week, every two days, every three days, every four days, every five days, every six days, weekly, bi-weekly, etc. In various aspects, the composition is administered to the subject once a week. The administration regimen for the first composition and an additional nanoparticle composition may be the same or may be different. For example, the administration regimen of an additional nanoparticle-containing composition, provided after CAR T cell administration, may occur at different intervals and for a longer period of time (i.e., a longer overall period of treatment) than the first composition. A composition comprising the CAR T cell may be administered according to the therapeutic regimen for the particular CAR T cell employed and cancer being treated.

The methods of the present disclosure may comprise the above described step(s) alone or in combination with other steps. The methods may comprise repeating any one of the above-described step(s) and/or may comprise additional steps, aside from those described above. For example, the presently disclosed methods may further comprise steps for making or preparing the nanoparticles or compositions of the present disclosure. For instance, the presently disclosed methods further comprise obtaining a sample of the tumor of the subject, optionally, via a biopsy. The methods also may further comprise isolating total RNA from the cells of the tumor, generating cDNA from the total RNA via reverse transcription, and amplifying mRNA from the cDNA. Optionally, the mRNA is incorporated into nanoparticles of, e.g., the second composition. The presently disclosed methods also in some aspects further comprise mixing the mRNA and the cationic lipid at a RNA:cationic lipid ratio of about 1 to about 10 to about 1 to about 20 (e.g., about 1 to about 19, about 1 to about 18, about 1 to about 17, about 1 to about 16, about 1 to about 15, about 1 to about 14, about 1 to about 13, about 1 to about 12, about 1 to about 11). In exemplary instances, the presently disclosed methods further comprise mixing the mRNA and the cationic lipid at a RNA:cationic lipid ratio of about 1 to about 15.

### Subjects

The subject is a mammal, including, but not limited to, mammals of the order Rodentia, such as mice and hamsters, and mammals of the order Logomorpha, such as rabbits, mammals from the order Carnivora, including Felines (cats) and Canines (dogs), mammals from the order Artiodactyla, including Bovines (cows) and Swines (pigs) or of the order Perssodactyla, including Equines (horses). In some aspects, the mammals are of the order Primates, Ceboids, or Simoids (monkeys) or of the order Anthropoids (humans and apes). In some aspects, the mammal is a human. In some aspects, the human is an adult aged 18 years or older. In some aspects, the human is a child aged 17 years or less.

In various aspects, the subject comprises a surface antigen negative solid tumor.

### Cancer

The cancer treatable by the methods disclosed herein may be any cancer, e.g., any malignant growth or tumor caused by abnormal and uncontrolled cell division that optionally may spread to other parts of the body through the lymphatic system or the blood stream. In various aspects, the subject has a solid tumor. In this regard, the disclosure provides a method of treating a solid tumor in a subject in need thereof, the method comprising administering to the subject a first composition comprising a nanoparticle comprising a positively-charged surface and an interior comprising (i) a core and (ii) at least two nucleic acid layers, wherein each nucleic acid layer is positioned between a cationic lipid bilayer, at least one day prior to administering CAR T cell therapy to the subject and, optionally, administering CAR T cell therapy thereafter. Also optionally, the method further comprises administering an additional (second) composition comprising a nanoparticle comprising a positively-charged surface and an interior comprising (i) a core and (ii) at least two nucleic acid layers, wherein each nucleic acid layer is positioned between a cationic lipid bilayer after administration of the CAR T cells.

The cancer in some aspects is one selected from the group consisting of acute lymphocytic cancer, acute myeloid leukemia, alveolar rhabdomyosarcoma, bone cancer, brain cancer (e.g., glioma), breast cancer (e.g., triple negative breast cancer), cancer of the anus, cancer of the anal canal, cancer of the anorectum, cancer of the eye, cancer of the intrahepatic bile duct, cancer of the joints, cancer of the head, neck, gallbladder, or pleura, cancer of the nose, nasal cavity, or middle ear, cancer of the oral cavity, cancer of the vulva, chronic lymphocytic leukemia, chronic myeloid cancer, colon cancer, esophageal cancer, cervical cancer, gastrointestinal cancer (e.g., gastrointestinal carcinoid tumor), Hodgkin lymphoma, endometrial or hepatocellular carcinoma, hypopharynx cancer, kidney cancer, larynx cancer, liver cancer, lung cancer (e.g., non-small cell lung cancer, bronchioloalveolar carcinoma), malignant mesothelioma, melanoma, multiple myeloma, nasopharynx cancer, non-Hodgkin lymphoma, ovarian cancer, osteosarcoma, pancreatic cancer, cancer of the peritoneum, cancer of the omentum, mesentery cancer, pharynx cancer, prostate cancer, rectal cancer, renal cancer (e.g., renal cell carcinoma (RCC)), small intestine cancer, soft tissue cancer, stomach cancer, testicular cancer, thyroid cancer, ureter cancer, and urinary bladder cancer. In particular aspects, the cancer is selected from the group consisting of head and neck, ovarian, cervical, bladder and oesophageal cancers, pancreatic, gastrointestinal cancer, gastric, breast, endometrial and colorectal cancers, hepatocellular carcinoma, glioblastoma, bladder and lung cancer (e.g., non-small cell lung cancer (NSCLC), bronchioloalveolar carcinoma). Optionally, the subject suffers from a malignant brain tumor, such as a glioblastoma, medulloblastoma, diffuse intrinsic pontine glioma, or a peripheral tumor with metastatic infiltration into the central nervous system. Optionally, the subject suffers from osteosarcoma, such as recurrent or metastatic osteosarcoma.

In various aspects, the subject is suffering from a refractory malignancy. In this respect, a tumor which evades a particular therapy or host immune response is “refractory” (or resistant). A tumor that is “sensitive” to a therapy demonstrates a beneficial clinical response to treatment. A tumor that is “sensitive” to a host immune response is recognized by the host immune system and subject to attack by immune effector cells.

In addition to preconditioning a subject for CAR T cell therapy, the RNA-LPs of the instant disclosure can transition an immunologically “cold” tumor, e.g., a tumor lacking infiltrating T cells and/or which is not recognized by the immune system, into an immunologically “hot” tumor, i.e., a tumor exhibiting, e.g., activated myeloid cells and/or lymphocyte infiltration and interferon production in the tumor microenvironment. Immunological treatment of “cold” tumors presents a great challenge due, at least in part, to the absence of an adaptive immune response. Cancers that tend to give rise to immunologically “cold” tumors include, but are not limited to, glioblastomas, ovarian cancer, prostate cancer, pancreatic cancer, and many breast cancers. “Cold” tumors are limited to these cancer types, however; as cancers evolve in a subject, some develop resistance mechanisms that allow evasion of the immune system. Surprisingly, the nanoparticles of the disclosure “reprogram” the tumor to be recognized by the host immune system.

In various aspects, the subject is suffering from an immune checkpoint inhibitor (ICI)-resistant malignancy. The susceptibility of a tumor to an immune response (or ICI) or, put another way, the effectiveness of an immune response (or ICI) against a tumor, can be determined in a variety of ways, such as those known in the art.

In some embodiments, the method described herein further comprises administration of one or more other therapeutic agents. In some aspects, the other therapeutic agent aims to treat or prevent cancer. In some embodiments, the other therapeutic is a chemotherapeutic agent. Common chemotherapeutics include, but are not limited to, adriamycin, asparaginase, bleomycin, busulphan, cisplatin, carboplatin, carmustine, capecitabine, chlorambucil, cytarabine, cyclophosphamide, camptothecin, dacarbazine, dactinomycin, daunorubicin, dexrazoxane, docetaxel, doxorubicin, etoposide, floxuridine, fludarabine, fluorouracil, gemcitabine, hydroxyurea, idarubicin, ifosfamide, irinotecan, lomustine, mechlorethamine, mercaptopurine, meplhalan, methotrexate, mitomycin, mitotane, mitoxantrone, nitrosurea, paclitaxel, pamidronate, pentostatin, plicamycin, procarbazine, rituximab, streptozocin, teniposide, thioguanine, thiotepa, vinblastine, vincristine, vinorelbine, taxol, transplatinum, 5-fluorouracil, and the like. The chemotherapy, if it is a lymphodepletion therapy, in various instances, is not administered within the timeframe prior to CAR T cell therapy, as described above.

In some embodiments, the other therapeutic is an agent used in radiation therapy for the treatment of cancer; indeed, in some embodiments, the method is part of a treatment regimen that includes radiation therapy. Further, the method of the disclosure can be performed in connection with surgical resection of a tumor, such as a glioma (e.g., glioblastoma).

In exemplary aspects, the method comprises administering an immune checkpoint inhibitor (ICI) to the subject. An “immune checkpoint inhibitor” or “ICI” is any agent (e.g., compound or molecule) that that decreases, blocks, inhibits, abrogates or interferes with the function of a protein of an immune checkpoint pathway. Proteins of the immune checkpoint pathway regulate immune responses and, in some instances, prevent T cells from attacking cancer cells. In various aspects, the protein of the immune checkpoint pathway is, for example, CTLA-4, PD-1, PD-L1, PD-L2, B7-H3, B7-H4, TIGIT, VISTA, LAG3, CD112 TIM3, BTLA, or co-stimulatory receptor ICOS, OX40, 41BB, or GITR. In various aspects, the ICI is a small molecule, an inhibitory nucleic acid, or an inhibitor polypeptide. In various aspects, the ICI is an antibody, antigen-binding antibody fragment, or an antibody protein product, that binds to and inhibits the function of the protein of the immune checkpoint pathway. Suitable ICIs which are antibodies, antigen-binding antibody fragments, or an antibody protein products are known in the art and include, but are not limited to, ipilimumab (CTLA-4; Bristol Meyers Squibb), nivolumab (PD-1; Bristol Meyers Squibb), pembrolizumab (PD-1; Merck), atezolizumab (PD-L1; Genentech), avelumab (PD-L1; Merck), and durvalumab (PD-L1; Medimmune) (Wei et al., Cancer Discovery 8: 1069-1086 (2018)). Other examples of ICIs include, but are not limited to, IMP321 (LAG3: Immuntep); BMS-986016 (LAG3; Bristol Meyers Squibb); IPH2101 (KIR; Innate Pharma); tremelimumab (CTLA-4; Medimmune); pidilizumab (PD-1; Medivation); MPDL3280A (PD-L1; Roche); MEDI4736 (PD-L1; AstraZeneca); MSB0010718C (PD-L1; EMD Serono); AUNP12 (PD-1; Aurigene); MGA271 (B7-H3: MacroGenics); and TSR-022 (TIM3; Tesaro).

### Methods of Nanoparticle Manufacture

The nanoparticle comprising a positively-charged surface and an interior comprising (i) a core and (ii) at least two nucleic acid (e.g., RNA) layers, wherein each nucleic acid layer is positioned between a cationic lipid bilayer, may be manufactured by a method comprising (A) mixing nucleic acid molecules and liposomes at a nucleic acid (e.g., RNA):liposome ratio of about 1 to about 5 to about 1 to about 25, such as about 1 to 5 to about 1 to about 20, optionally, about 1 to about 15, to obtain nucleic acid- (e.g., RNA-) coated liposomes. The liposomes are made by a process of making liposomes comprising drying a lipid mixture comprising a cationic lipid and an organic solvent by evaporating the organic solvent under a vacuum. The method further comprises (B) mixing the RNA-coated liposomes with a surplus amount of liposomes. In exemplary aspects, the nanoparticle made by the presently disclosed method accords with the descriptions of the nanoparticles described herein. For example, the nanoparticle made by the presently disclosed methods has a zeta potential of about +40 mV to about +60 mV, optionally, about +45 mV to about +55 mV. Optionally, the zeta potential of the nanoparticle made by the presently disclosed methods is about +50 mV. In various aspects, the core of the nanoparticle made by the presently disclosed methods comprises less than about 0.5 wt % nucleic acid and/or the core comprises a cationic lipid bilayer and/or the outermost layer of the nanoparticle comprises a cationic lipid bilayer and/or the surface of the nanoparticle comprises a plurality of hydrophilic moieties of the cationic lipid of the cationic lipid bilayer.

In exemplary aspects, the lipid mixture comprises the cationic lipid and the organic solvent at a ratio of about 40 mg cationic lipid per mL organic solvent to about 60 mg cationic lipid per mL organic solvent, optionally, at a ratio of about 50 mg cationic lipid per mL organic solvent. In various instances, the process of making liposomes further comprises rehydrating the lipid mixture with a rehydration solution to form a rehydrated lipid mixture and then agitating, resting, and sizing the rehydrated lipid mixture. Optionally, sizing the rehydrated lipid mixture comprises sonicating, extruding and/or filtering the rehydrated lipid mixture.

A description of an exemplary method of making a nanoparticle is provided herein at Example 1. It will be appreciated that any one or more of the steps described in Example 1 may be adjusted as needed. For instance, in some embodiments, the method comprises one or more steps required for preparing the RNA prior to being complexed with the liposomes. In exemplary aspects, downstream steps are included to prepare the nanoparticles for administration to a subject, e.g., a human. In exemplary instances, the method comprises formulating the NP for intravenous injection. The method comprises in various aspects adding one or more pharmaceutically acceptable carriers, diluents, or excipients, and optionally comprises packaging the resulting composition in a container, e.g., a vial, a syringe, a bag, an ampoule, and the like. The container in some aspects is a ready-to-use container and optionally is for single-use.

### Pharmaceutical Compositions

Provided herein are compositions comprising a nanoparticle of the present disclosure and a pharmaceutically acceptable carrier, excipient or diluent. In exemplary aspects, the composition is a sterile composition. In exemplary instances, the composition comprises a plurality of nanoparticles of the present disclosure. Optionally, at least 50% of the nanoparticles of the plurality have a diameter between about 100 nm to about 250 nm. In various aspects, the composition comprises about 10¹⁰ nanoparticles per mL to about 10¹⁵ nanoparticles per mL, optionally about 10¹² nanoparticles±10% per mL.

In exemplary aspects, the composition of the present disclosure may comprise additional components other than the nanoparticle, a cell comprising the nanoparticle, a population of cells comprising the nanoparticle, or CAR T cell. The composition, in various aspects, comprises any pharmaceutically acceptable ingredient, including, for example, acidifying agents, additives, adsorbents, aerosol propellants, air displacement agents, alkalizing agents, anticaking agents, anticoagulants, antimicrobial preservatives, antioxidants, antiseptics, bases, binders, buffering agents, chelating agents, coating agents, coloring agents, desiccants, detergents, diluents, disinfectants, disintegrants, dispersing agents, dissolution enhancing agents, dyes, emollients, emulsifying agents, emulsion stabilizers, fillers, film forming agents, flavor enhancers, flavoring agents, flow enhancers, gelling agents, granulating agents, humectants, lubricants, mucoadhesives, ointment bases, ointments, oleaginous vehicles, organic bases, pastille bases, pigments, plasticizers, polishing agents, preservatives, sequestering agents, skin penetrants, solubilizing agents, solvents, stabilizing agents, suppository bases, surface active agents, surfactants, suspending agents, sweetening agents, therapeutic agents, thickening agents, tonicity agents, toxicity agents, viscosity-increasing agents, water-absorbing agents, water-miscible cosolvents, water softeners, or wetting agents. See, e.g., the Handbook of Pharmaceutical Excipients, Third Edition, A. H. Kibbe (Pharmaceutical Press, London, U K, 2000), which is incorporated by reference in its entirety. Remington's Pharmaceutical Sciences, Sixteenth Edition, E. W. Martin (Mack Publishing Co., Easton, Pa., 1980), which is incorporated by reference in its entirety.

Compositions of the present disclosure can be suitable for administration by any acceptable route, including parenteral and subcutaneous. Other routes include intravenous, intradermal, intramuscular, intraperitoneal, intranodal and intrasplenic, for example. In exemplary aspects, the composition is suitable for systemic (e.g., intravenous) administration. If the composition is in a form intended for administration to a subject, it can be made to be isotonic with the intended site of administration. For example, if the solution is in a form intended for administration parenterally, it can be isotonic with blood. The composition typically is sterile. In certain embodiments, this may be accomplished by filtration through sterile filtration membranes. In certain embodiments, parenteral compositions generally are placed into a container having a sterile access port, for example, an intravenous solution bag, or vial having a stopper pierceable by a hypodermic injection needle, or a prefilled syringe. In certain embodiments, the composition may be stored either in a ready-to-use form or in a form (e.g., lyophilized) that is reconstituted or diluted prior to administration.

### EXAMPLES

The following examples are given merely to illustrate the present invention and not in any way to limit its scope.

### Example 1

This example describes a method of making nanoparticles of the present disclosure.

### Preparation of DOTAP Liposomes

On Day 1, the following steps were carried out in the fume hood. Water was added to a rotavapor bath. Chloroform (20 mL) was poured into a sterile, glass graduated cylinder. After opening a vial containing 1 g of DOTAP, 5 mL chloroform was added to the DOTAP vial using a glass pipette. The volume of chloroform and DOTAP was then transferred into a 1-L evaporating flask. The DOTAP vial was washed by adding a second 5-mL volume of chloroform to the DOTAP vial to dissolve any remaining DOTAP in the vial and then transferring this volume of chloroform from the DOTAP vial to the evaporating flask. This washing step was repeated 2 more times until all the chloroform in the graduated cylinder was used. The evaporating flask was then placed into the Buchi rotavapor. The water bath was turned on and adjusted to 25° C. The evaporating flask was moved downward until it touched the water bath. The rotation speed of the rotavapor was adjusted to 2. The vacuum system was turned on and adjusted to 40 mbar. After 10 minutes, the vacuum system was turned off and the chloroform was collected from the collector flask. The amount of chloroform collected was measured. Once the collector flask is repositioned, the vacuum was turned on again and the contents in the evaporating flask was allowed to dry overnight until the chloroform was completely evaporated.

On Day 2, using a sterile graduated cylinder, PBS (200 mL) was added to a new, sterile 500-mL PBS bottle maintained at room temperature. A second 500-mL PBS bottle was prepared for collecting DOTAP. The Buchi rotavapor water bath was set to 50° C. PBS (50 mL) was added into the evaporating flask using a 25-mL disposable serological pipette. The evaporating flask was positioned in the Buchi rotavapor and moved downward until ⅓ of the flask was submerged into the water bath. The rotation speed of the rotavapor was set to 2, allowed to rotate for 10 min, and then rotation was turned off. A 50-mL volume of PBS with DOTAP from the evaporating flask was transferred to the second 500 mL PBS bottle. The steps were repeated (3-times) until the entire volume of PBS in the PBS bottle was used. The final volume of the second 500 mL PBS bottle was 400 mL. The lipid solution in the second 500 mL PBS bottle was vortexed for 30 s and then incubated at 50 ºC for 1 hour. During the 1 hour incubation, the bottle was vortexed every 10 min. The second 500 mL PBS bottle was allowed to rest on overnight at room temperature.

On Day 3, PBS (200 mL) was added to the second 500 mL PBS bottle containing DOTAP and PBS. The second 500 mL PBS bottle was placed into an ultrasonic bath. Water was filled in the ultrasonic bath and the second 500 mL PBS bottle was sonicated for 5 min. The extruder was washed with PBS (100 mL) and this wash step was repeated. A 0.45 μm pore filter was assembled into a filtration unit and a new (third) 500 mL PBS bottle was positioned into the output tube of the extruder. In a biological safety cabinet, the DOTAP-PBS mixture was loaded into the extruder, until about 70% of the third PBS bottle was filled. The extruder was then turned on and the DOTAP PBS mixture was added until all the mixture was run through the extruder. Subsequently, a 0.22 μm pore filter was assembled into the filtration unit and a new (third) 500 mL PBS bottle was positioned into the output tube of the extruder. The previously filtered DOTAP-PBS mixture was loaded and run again throughout. The samples comprising DOTAP lipid nanoparticles (NPs) in PBS were then stored at 4 ºC.

### RNA Preparation

Prior to incorporation into NPs, RNA was prepared in one of a few ways. Total tumor RNA was prepared by isolating total RNA (including rRNA, tRNA, mRNA) from tumor cells. In vitro transcribed mRNA was prepared by carrying out in vitro transcription reactions using cDNA templates produced by reverse transcription of total tumor RNA. Tumor antigen-specific and Non-specific RNAs were either made in-house or purchased from a vendor.

Total Tumor RNA: Total tumor-derived RNA from tumor cells (e.g., B16F0, B16F10, and KR158-luc) is isolated using commercially available RNeasy mini kits (Qiagen) based on manufacturer instructions.

In vitro transcribed mRNA: Briefly, RNA is isolated using commercially available RNeasy mini kits (Qiagen) per manufacturer's instructions and cDNA libraries were generated by RT-PCR. Using a SMARTScribe Reverse Transcriptase kit (Takara), a reverse transcriptase reaction by PCR was performed on the total tumor RNA in order to generate cDNA libraries. The resulting cDNA was then amplified using Takara Advantage 2 Polymerase mix with T7/SMART and CDS III primers, with the total number of amplification cycles determined by gel electrophoresis. Purification of the cDNA was performed using a Qiagen PCR purification kit per manufacturer's instructions. In order to isolate sufficient mRNA for use in each RNA-nanoparticle vaccine, mMESAGE mMACHINE (Invitrogen) kits with T7 enzyme mix were used to perform overnight in vitro transcription on the cDNA libraries. Housekeeping genes were assessed to ensure fidelity of transcription. The resulting mRNA was then purified with a Qiagen RNeasy Maxi kit to obtain the final mRNA product.

Tumor Antigen-Specific and Non-Specific mRNA: Plasmids comprising DNA encoding tumor antigen-specific RNA (RNA encoding, e.g., pp65, OVA) and non-specific RNA (RNA encoding, e.g., Green Fluorescent Protein (GFP), luciferase) are linearized using restriction enzymes (i.e., SpeI) and purified with Qiagen PCR MiniElute kits. Linearized DNA is subsequently transcribed using the mmRNA in vitro transcription kit (Life technologies, Invitrogen) and cleaned up using RNA Maxi kits (Qiagen). In alternative methods, non-specific RNA is purchased from Trilink Biotechnologies (San Diego, CA).

### Preparation of Multilamellar RNA Nanoparticles (NPs)

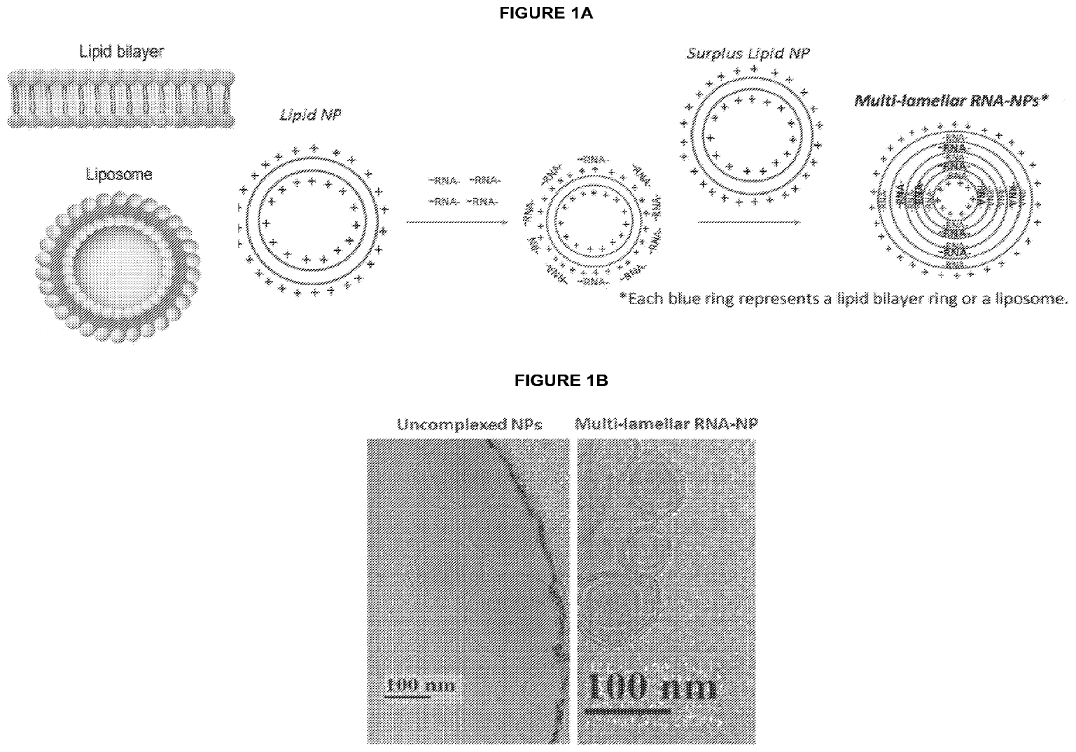

The DOTAP lipid NPs were complexed with RNA to make multilamellar RNA-NPs which were designed to have several layers of mRNA contained inside a tightly coiled liposome with a positively charged surface and an empty core (FIG. 1A). Briefly, in a safety cabinet, RNA was thawed from −80° C. and then placed on ice, and samples comprising PBS and DOTAP (e.g., DOTAP lipid NPs) were brought up to room temperature. Once components were prepared, the desired amount of RNA was mixed with PBS in a sterile tube. To the sterile tube containing the mixture of RNA and PBS, the appropriate amount of DOTAP lipid NPs was added without any physical mixing (without e.g., inversion of the tube, without vortexing, without agitation). The mixture of RNA, PBS, and DOTAP was incubated for about 15 minutes to allow multilamellar RNA-NP formation. After 15 min, the mixture was gently mixed by repeatedly inverting the tube. The mixture was then considered ready for systemic (i.e. intravenous) administration.

The amount of RNA and DOTAP lipid NPs (liposomes) used in the above preparation is pre-determined or pre-selected. In some instances, a ratio of about 15 μg liposomes per about 1 μg RNA were used. For instance, about 75 μg liposomes are used per ˜5 μg RNA or about 375 μg liposomes are used per ˜25 μg RNA. In other instances, about 7.5 μg liposomes were used per 1 μg RNA. Thus, in exemplary instances, about 1 μg to about 20 μg liposomes are used for every μg RNA used.

### Example 2

This example describes the characterization of the nanoparticles of the present disclosure.

### Cryo-Electron Microscopy (CEM)

CEM was used to analyze the structure of multilamellar RNA-NPs prepared as described in Example 1 and control NPs devoid of RNA (uncomplexed NPs) which were made by following all the steps of Example 1, except for the steps under “RNA Preparation” and “Preparation of Multilamellar RNA nanoparticles (NPs)”. CEM was carried out as essentially described in Sayour et al., Nano Lett 17(3) 1326-1335 (2016). Briefly, samples comprising multilamellar RNA-NPs or control NPs were kept on ice prior to being loaded in a snap-frozen in Vitrobot (and automated plunge-freezer for cryoTEM, that freezes samples without ice crystal formation, by controlling temperature, relative humidity, blotting conditions and freezing velocity). Samples were then imaged in a Tecnai G2 F20 TWIN 200 kV/FEG transmission electron microscope with a Gatan UltraScan 4000 (4k×4k) CCD camera. The resulting CEM images are shown in FIG. 1B. The right panel is a CEM image of multilamellar RNA-NPs and the left panel is a CEM image of control NPs (uncomplexed NPs). As shown in FIG. 1B, the control NPs contained at most 2 layers, whereas multilamellar RNA NPs contained several layers.

### Zeta Potentials

Zeta potentials of multilamellar RNA NPs were measured by phase analysis light scattering (PALS) using a Brookhaven ZetaPlus instrument (Brookhaven Instruments Corporation, Holtsville, NY), as essentially described in Sayour et al., Nano Lett 17(3) 1326-1335 (2016). Briefly, uncomplexed NPs or RNA-NPs (200 μL) were resuspended in PBS (1.2 mL) and loaded in the instrument. The samples were run at 5 runs per sample, 25 cycles each run, and using the Smoluchowski model.

The zeta potential of the multilamellar RNA NPs prepared as described in Example 1 was measured at about +50 mV. Interestingly, this zeta potential of the multilamellar RNA NPs was much higher than those described in Sayour et al., Oncoimmunology 6(1): e1256527 (2016), which measured at around +27 mV. Without being bound to any particular theory, the way in which the DOTAP lipid NPs are made for use in making the multilamellar RNA NPs (Example 1) involving a vacuum-seal method for evaporating off chloroform leads to less environmental oxidation of the DOTAP lipid NPs, which, in turn, may allow for a greater amount of RNA to complex with the DOTAP NPs and/or greater incorporation of RNA into the DOTAP lipid NPs.

### RNA Incorporation by Gel Electrophoresis:

A gel electrophoresis experiment was conducted to measure the amount of RNA incorporated into ML liposomes. Based on this experiment, it was qualitatively shown that nearly all, if not all, of the RNA used in the procedure described in Example 1 was incorporated into the DOTAP lipid NPs. Additional experiments to characterize the extent of RNA incorporation are carried out by measuring RNA-NP density and comparing this parameter to that of lipoplexes.

### Example 3

This example describes a comparison of the nanoparticles of the present disclosure to cationic RNA lipoplexes and anionic RNA lipoplexes.

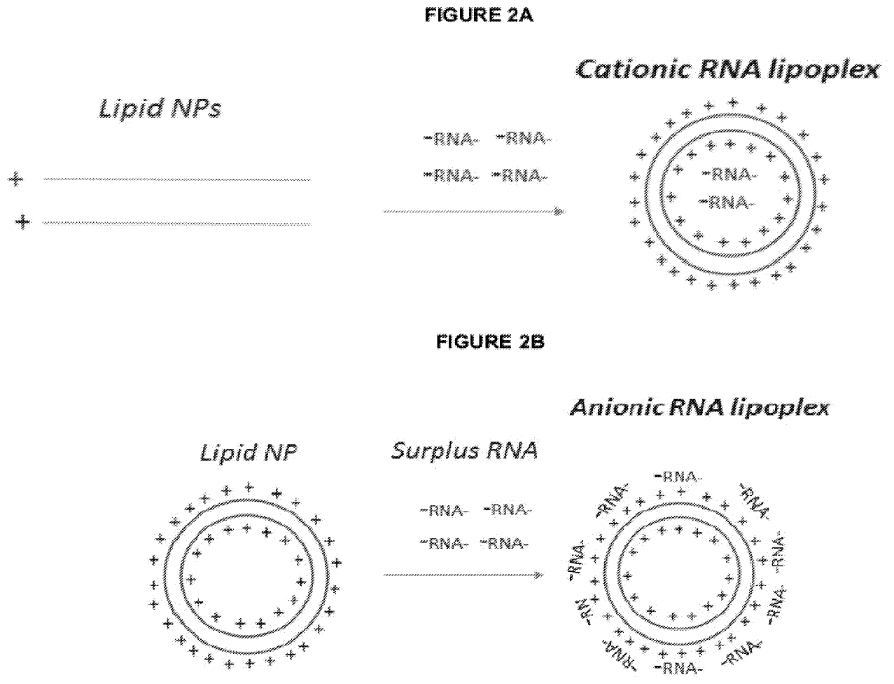

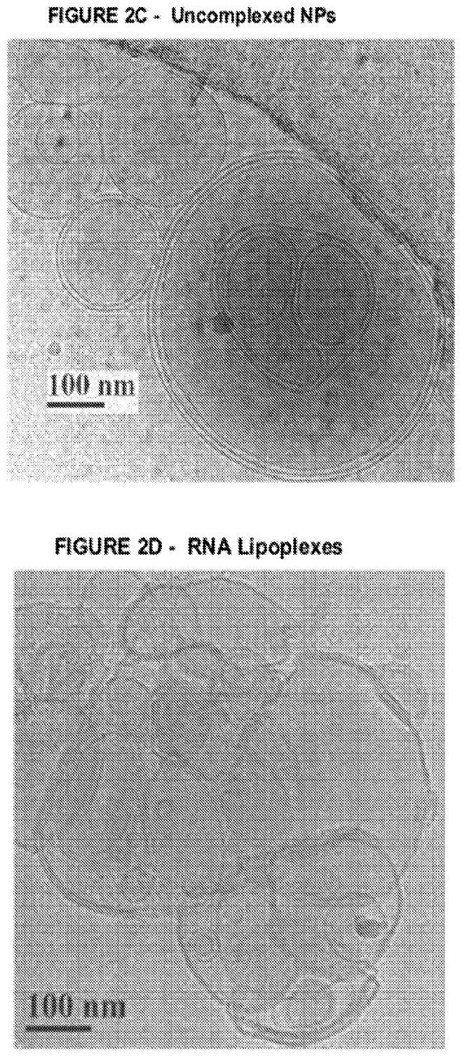

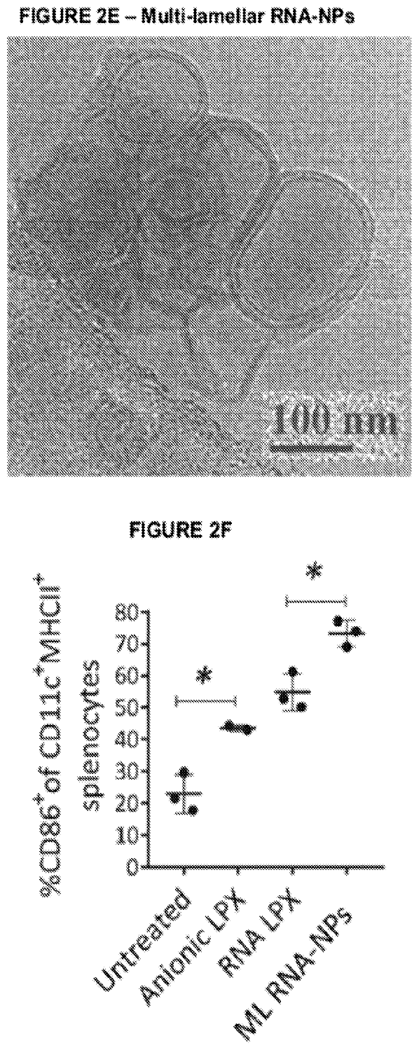

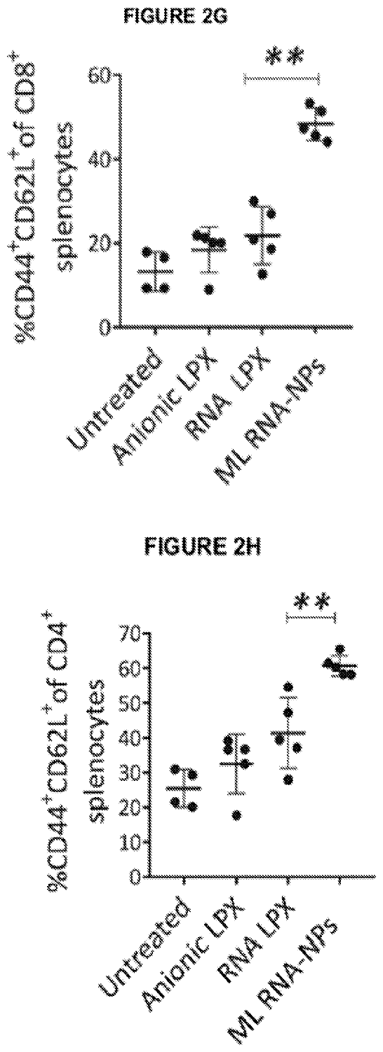

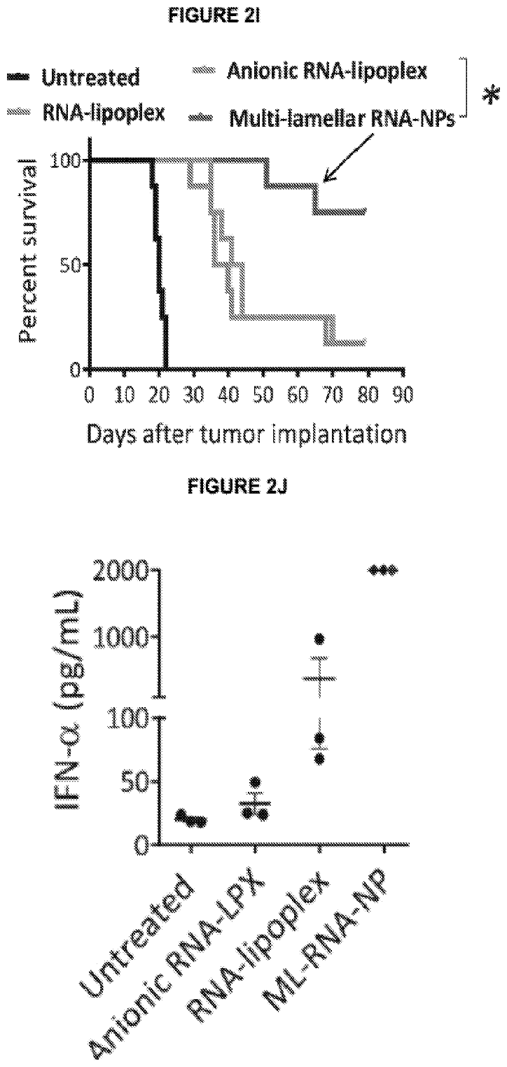

Cationic lipoplexes (LPX) were first developed with mRNA in the lipid core shielded by a net positive charge located on the outer surface (FIG. 2A). Anionic RNA lipoplexes (FIG. 2B) have been developed with an excess of RNA tethered to the surface of bi-lamellar liposomes. RNA-LPX were made by mixing RNA and lipid NP at ratios to equalize charge. Anionic RNA-NPs were made by mixing RNA and lipid NP at ratios to oversaturate lipid NPs with negative charge. Various aspects of the RNA-LPX and anionic RNA LPX were then compared to the multilamellar RNA NPs described in the above examples.

Cryo-Electron Microscopy (CEM) was used to compare the structures of the RNA LPX and the multilamellar RNA-NPs prepared as described in Example 1. Uncomplexed NPs were used as a control. CEM was carried out as essentially described in Example 2. FIG. 2C is a CEM image of uncomplexed NPs, FIG. 2D is a CEM image of RNA LPXs (wherein that mass ratio of liposome to RNA is 3.75:1) and FIG. 2E is a CEM image of the multilamellar RNA-NPs (wherein that mass ratio of liposome to RNA is 15:1). These data support that more RNA is held by the ML RNA-NPs. Additional data show that the concentration drops more with ML RNA-NP complexation versus RNA LPX supporting multilamellar formation of ML RNA-NPs not observed by simple mixing of equivalent amounts of RNA and lipid NPs by mass or charge (i.e. RNA-LPX and anionic RNA-LPX respectively). This supports that more RNA is “held” by ML RNA-NPs described herein.

Also, an experiment was conducted to determine where the anionic LPXs localize upon administration to mice. Anionic LPXs localized to the spleens of animals upon administration.

RNA LPX, anionic lipoplex (LPX) or multilamellar RNA-NPs were administered to mice and spleens were harvested one week later for assessment of activated DCs (*p<0.05 unpaired t test). The RNA used in this experiment was tumor-derived mRNA from the K7M2 tumor osteosarcoma cell line. As shown in FIG. 2F, mice treated with multilamellar RNA NPs exhibited the highest levels of activated DCs.

Anionic tumor mRNA-lipoplexes, tumor mRNA-lipoplexes, and multilamellar tumor mRNA loaded NPs were compared in a therapeutic lung cancer model (K7M2) (n=5-8/group). Each vaccine was intravenously administered weekly (×3) (** p<0.01, Mann Whitney). The % CD44+CD62L+ of CD8+ splenocytes is shown in FIG. 2G and the % CD44+CD62L+ of CD4+ splenocytes is shown in FIG. 2H. Also, FIG. 2J shows that multilamellar (ML) RNA-NPs mediate substantially increased IFN-alpha, which is an innate anti-viral cytokine. This demonstrates that ML RNA-NPs allow for substantially greater innate immunity which is enough to drive efficacy from even non-antigen specific ML RNA-NPs. These data also indirectly support that ML RNA-NPs increase the number of activated plasmacytoid dendritic cells (pDCs) which cells are the most important producers of IFN-alpha. Taken together, the data demonstrates the superior efficacy of multilamellar tumor specific RNA-NPs, relative to anionic LPX and RNA LPX.

Anionic tumor mRNA-lipoplexes, cationic tumor mRNA-lipoplexes and multilamellar tumor mRNA loaded NPs were compared in a therapeutic lung cancer model (K7M2) (n=8/group). Each vaccine was iv administered weekly (×3), *p<0.05, Gehan Breslow-Wilcoxon test. The percent survival was measured by Kaplan-Meier Curve analysis. As shown in FIG. 2I, multilamellar tumor specific RNA-NPs mediated superior efficacy, compared to cationic RNA lipoplexes and anionic RNA lipoplexes, for increasing survival.

The ability of multilamellar RNA-NP to activate the innate immune response in vivo also was examined in the glioma tumor microenvironment.

RNA-NPs localize to perivascular regions of tumors and reprogram the TME in favor of activated myeloid cells. K-luc bearing animals (n=5/group) were vaccinated with tumor RNA-NPs or NPs alone. Tumors were harvested 48 h later for RNA-seq analysis. In animals receiving RNA-NPs, a significant upregulation of gene signatures for BATF3, IRFs, and IFN response genes was observed. In particular, the RNA-NP of the invention significantly upregulated expression of BATF3 (associated with effector dendritic cell phenotype), IRF5 and IRF7 (interferon regulatory factors), and ISG15 and IFITM3 (interferon response genes). These genes have been shown to be essential for sensitizing immunotherapeutic responses. As such, the RNA-NPs upregulate critical innate immune gene signatures in the glioma tumor microenvironment that associated with effector immune response, in effect turning tumors from “cold” to “hot,” allowing immune checkpoint inhibitors to be active where they were previously ineffective prior to RNA-NP treatment.

Herein it is demonstrated that the multilamellar RNA-NP formulation targeting physiologically relevant tumor antigens is more immunogenic (FIGS. 2F-2H, 2J) and significantly more efficacious (FIG. 2I) compared with anionic LPX and RNA LPX. Without being bound to any particular theory, by altering RNA-lipid ratios and increasing the zeta potential, a novel RNA-NP design composed of multi-lamellar rings of tightly coiled mRNA has been developed (FIG. 1C), which multi-lamellar design is thought to facilitate increased NP uptake of mRNA (condensed by alternating positive/negative charge) for enhanced particle immunogenicity and widespread in vivo localization to the periphery and tumor microenvironment (TME). Systemic administration of these multi-lamellar RNA-NPs localize to lymph nodes, reticuloendothelial organs (i.e. spleen and liver) and to the TME, activating DCs therein (based on increased expression of the activation marker CD86 on CD11c+ cells). These activated DCs prime antigen specific T cell responses, which lead to anti-tumor efficacy (with increased TILs) in several tumor models.

### Example 4

This example demonstrates personalized tumor RNA-NPs are active in a translational canine model.

The safety and activity of multilamellar RNA-NPs was evaluated in client-owned canines (pet dogs) diagnosed with malignant gliomas or osteosarcomas. The malignant gliomas or osteosarcomas from dogs were first biopsied for generation of personalized tumor RNA-NP vaccines.

To generate personalized multilamellar RNA NPs, total RNA materials was extracted from each patient's biopsy. A cDNA library was then prepared from the extracted total RNA, and then mRNA was amplified from the cDNA library. mRNA was then complexed with DOTAP lipid NPs, into multilamellar RNA-NPs as essentially described in Example 1. Blood was drawn at baseline, then 2 hours and 6 hours post-vaccination for assessment of, PD-L1, MHCII, CD80, and CD86 on CD11c+ cells. CD11c expression of PD-L1, MHC-II, PDL1/CD80, and PD-L1/CD86 is plotted over time during the canine's initial observation period. CD3+ cells were analyzed over time during the canine's initial observation period for percent CD4 and CD8, and these subsets were assessed for expression of activation markers (i.e., CD44). From these data, it was shown that multilamellar RNA-NPs elicited an increase in 1) CD80 and MHCII on CD11c⁺ peripheral blood cells demonstrating activation of peripheral DCs; and 2) an increase in activated T cells

Interestingly, within a few hours after administration, tumor specific RNA-NPs elicited margination of peripheral blood mononuclear cells, which increased in the subsequent days and weeks post-treatment, suggesting that RNA-NPs mediate lymphoid honing of immune cell populations before egress.

These data demonstrated that personalized mRNA-NPs are safe and active in translational canine disease models.

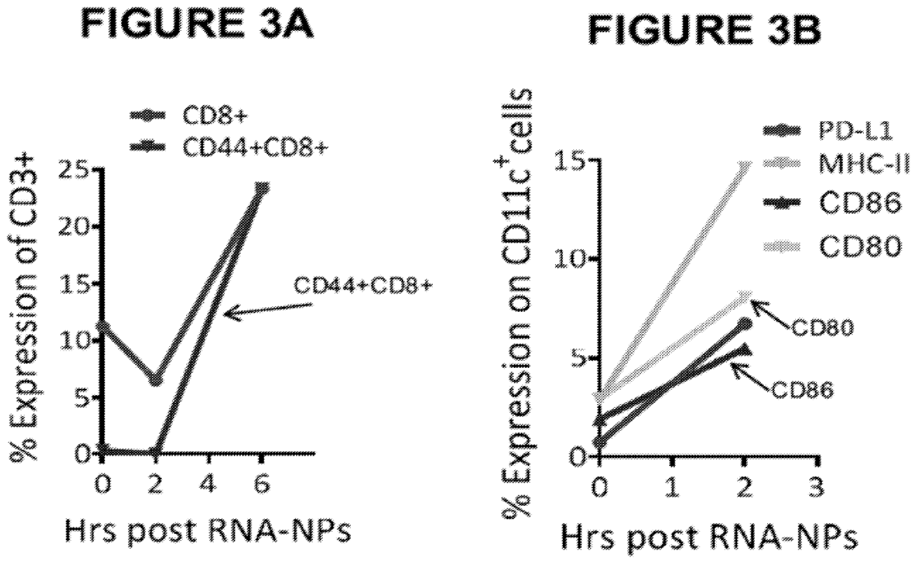

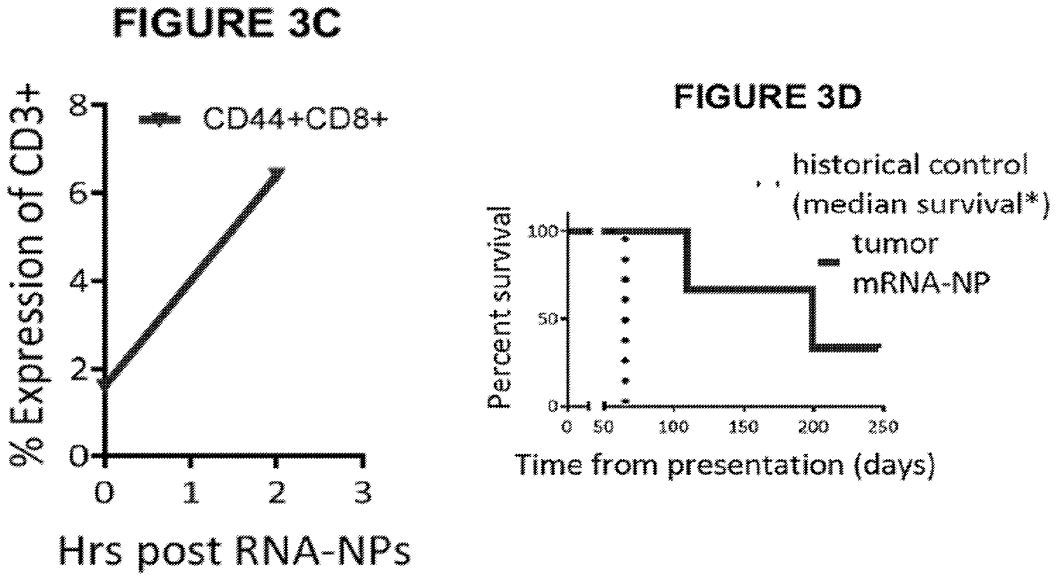

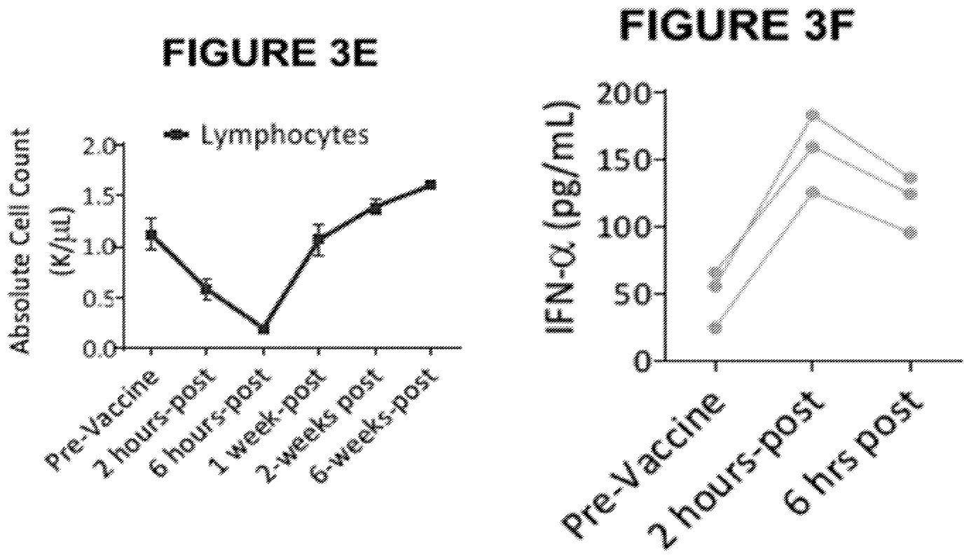

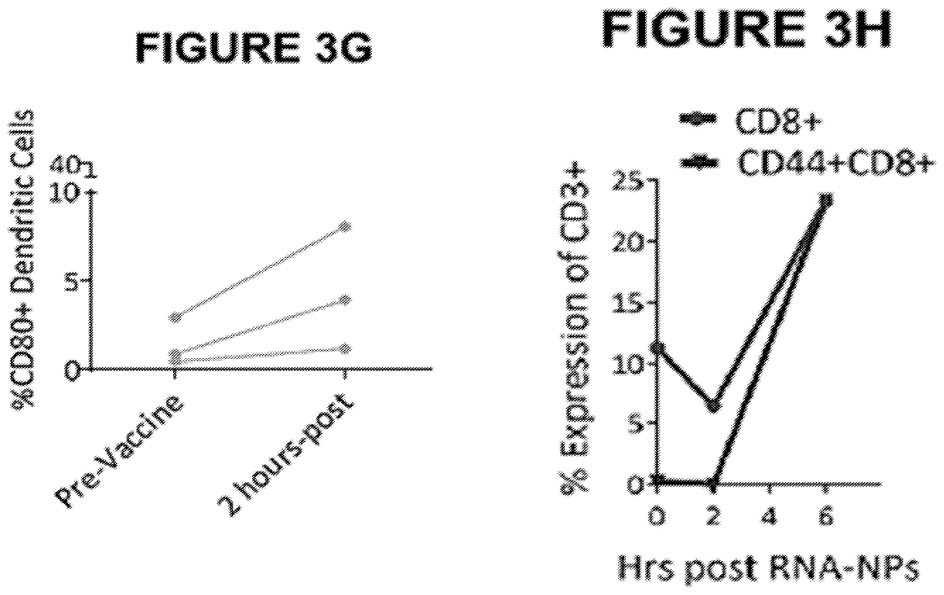

Specific data from canines evaluated in this manner are shown. A 31 kg male Irish Setter was enrolled on study per owner's consent to receive multilamellar RNA-NPs. Tumor mRNA was successfully extracted and amplified after tumor biopsy. Immunologic response was plotted in response to 1^{st} vaccine. The data show increased activation markers over time on CD11c+ cells (DCs) (FIG. 3A) The data show increased CD8+ cells that are activated (CD44+CD8+ cells) within the first few hours post RNA-NP vaccine. These data support that the multilamellar RNA-NPs are immunologically active in a male Irish Setter. A male boxer diagnosed with a malignant glioma was enrolled on study per owner's consent to receive RNA-NPs. Tumor mRNA was successfully extracted and amplified after tumor biopsy. Immunologic response is plotted in response to 1st vaccine (FIG. 3B). The data show increased activation markers over time on CD11c+ cells (DCs). As shown in FIG. 3C, an increase in activated T cells (CD44+CD8+ cells) was observed within the first few hours post RNA-NP vaccine. These data support that the multilamellar RNA-NPs are immunologically active in a male canine boxer. Additional observations from treatment of canines with spontaneous glioma are illustrated in FIGS. 3E-3H. FIG. 3E illustrates the percentage of lymphocytes elicited in the days post-vaccination, which suggests margination for antigen education before egress. FIG. 3F illustrates a spike in interferon-α production, and FIG. 3G illustrates an increase in CD80 expression in CD11c+ cells, in the hours following administration of the ML RNA-NPs. FIG. 3H illustrates expression of CD8+ cells and CD44+CD8+ cells, noting a shift toward a more immunologically “active” environment. The data support the use of ML RNA-NPs to transition toward an immune milieu that is more responsive to immunotherapy.

After receiving weekly RNA-NPs (×3), the canines diagnosed with malignant gliomas had a steady course. Post vaccination MRI showed stable tumor burdens, with increased swelling and enhancement (in some cases), which may be more consistent with pseudoprogression from an immunotherapeutic response in otherwise asymptomatic canines. Survival of canines diagnosed with malignant gliomas receiving only supportive care and tumor specific RNA-NPs (following tumor biopsy without resection) is shown in FIG. 3D. In FIG. 3D, the median survival (shown as dotted line) was about 65 days and was reported from a meta-analysis of canine brain tumor patients receiving only symptomatic management. In a previous study, cerebral astrocytomas in canines has been reported to have a median overall survival of 77 days. The personalized, multilamellar RNA NPs allowed for survival past 200 days.

Aside from low-grade fevers that spiked 6 hrs post-vaccination on the initial day, personalized tumor RNA-NPs (1×) were well tolerated with stable blood counts, differentials, renal and liver function tests. To date, four canines diagnosed with malignant brain tumors were treated. It is important to highlight that these canines received no other therapeutic interventions for their malignancies (i.e., no surgery, radiation or chemotherapy), and all patients assessed developed immunologic response with pseudoprogression or stable/smaller tumors. One canine was autopsied after RNA-NP vaccines. In this patient there were no toxicities believed to be related to the interventional agent.

These results suggest safety and activity of tumor specific RNA-NPs in client-owned canines with malignant brain tumors for subjects that did not receive any other anti-tumor therapeutic interventions.

### Example 5

This example demonstrates non-antigen specific multilamellar (ML) RNA NPs mediate antigen-specific immunity long enough to confer memory and fend off re-challenge of tumor.

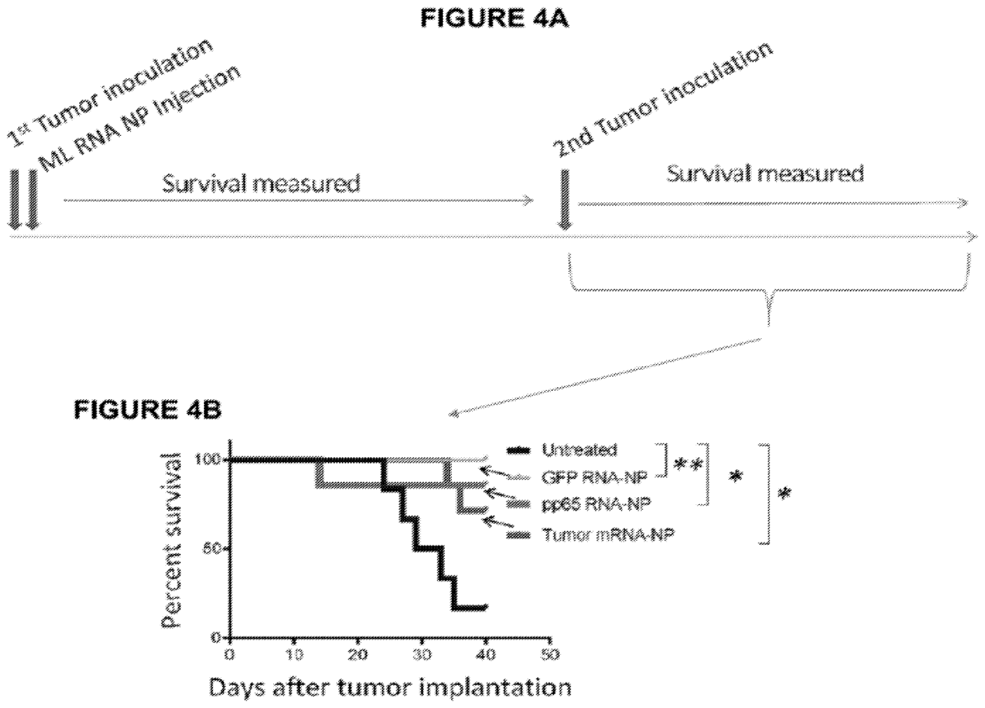

An experiment was carried out with long-term surviving mice (e.g., mice that survived for ˜100 days) that were challenged a total of two times via tumor inoculation, but treated only once weekly (×3) with ML RNA NPs comprising GFP RNA or pp65 RNA (each of which were non-specific to the tumor) or with ML RNA NPs comprising tumor-specific RNA. The treatment occurred just after the first tumor inoculation and about 100 days before the second tumor inoculation. Because none of the control mice (untreated mice) survived to 100 days, a new control group of mice were created by inoculating the same type of mice with K7M2 tumors. The new control group like the original control mice did not receive any treatment. The long-time survivors also did not receive any treatment after the second time of tumor inoculation. A timeline of the events of this experiment are depicted in FIG. 4A.

Remarkably, mice in all 3 groups contained long-time survivors that survived the second tumor challenge. As shown in FIG. 4B (which shows only the time period following the 2^{nd} inoculation), mice in all 3 groups contained long-time survivors with survival to 40 days post tumor implantation (second instance of tumor inoculation). Interestingly, the percentage of long-time survivor mice that were previously treated with ML RNA NPs comprising non-specific RNA (GFP RNA or pp65 RNA) survived to 40 days post second tumor inoculation, comparable to the group treated with ML RNA NPs comprising tumor specific RNA (treated before second tumor challenge).

These data support that ML RNA NPs comprising RNA non-specific to a tumor in a subject provides therapeutic treatment for the tumor comparable to that provided by ML RNA NPs comprising RNA specific to the tumor, leading to increased percentage in animal survival.

### Example 6

This example demonstrates that the administration of ML RNA NPs in combination with an immune checkpoint inhibitor leads to significantly increased survival in tumor-bearing subjects.

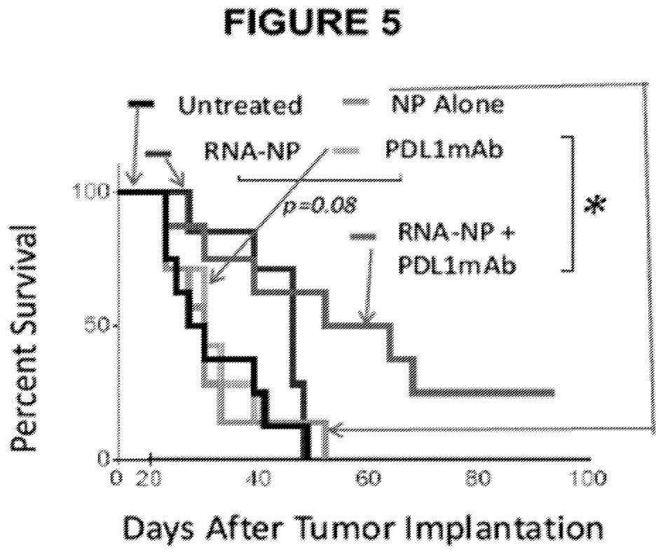

To test the effect of ML RNA NPs in combination with an ICI, tumor bearing C57Bl/6 mice were treated with ML RNA NPs alone (RNA NPs) or in combination with an anti-PDL1 monoclonal antibody (PDL1 mAb). Control groups included untreated mice, mice treated with nanoparticles not loaded with any RNA (NPs alone) or with just PDL1 mAb. For tumor implantation, ˜200,000 MOC-1 cells, which are mouse oral cavity squamous cell carcinoma (OSCC) cells were implanted subcutaneously in C57Bl/6 mice. For the groups receiving nanoparticles (ML RNA NPs alone or in combination with PDL1 mAb or NPs Alone), the NPs were injected intravenously within 24 hours of tumor implantation and then two more times once weekly. For the groups of mice receiving ICI (ML RNA NPs+PDL1 mAb or PDL1 mAb alone), PD-L1 mAbs (400 μg) were injected intraperitoneally followed by 200 μg twice weekly until the third dose of NPs was administered. Surviving mice from each group were monitored over the study period of about 100 days and the percentage of mice in each group surviving was plotted as a function of time post tumor implantation. The results are shown in FIG. 5. As shown in this figure, the percentage of surviving mice treated with ML RNA NPs in combination with an ICI was far greater than those receiving either treatment alone.

### Example 7

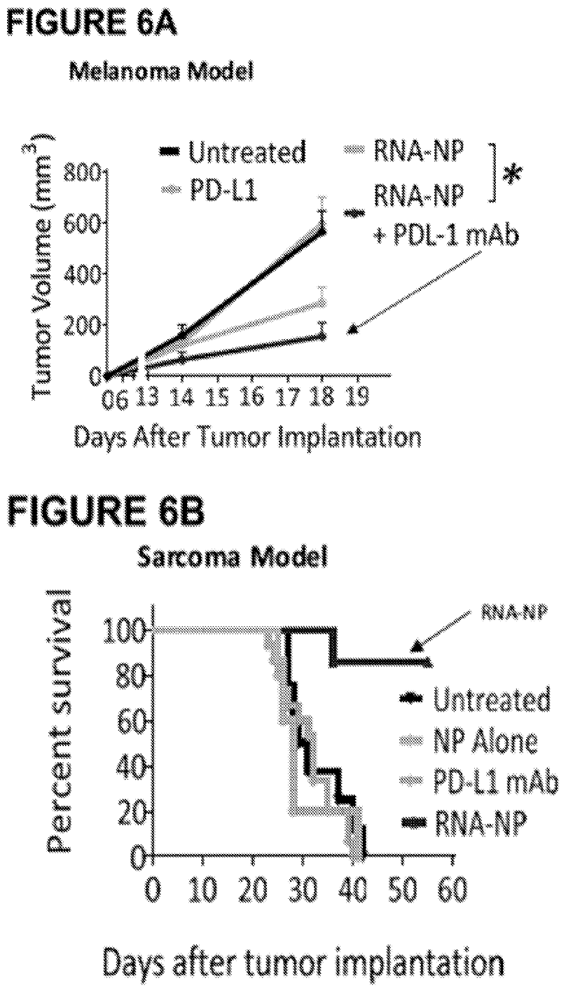

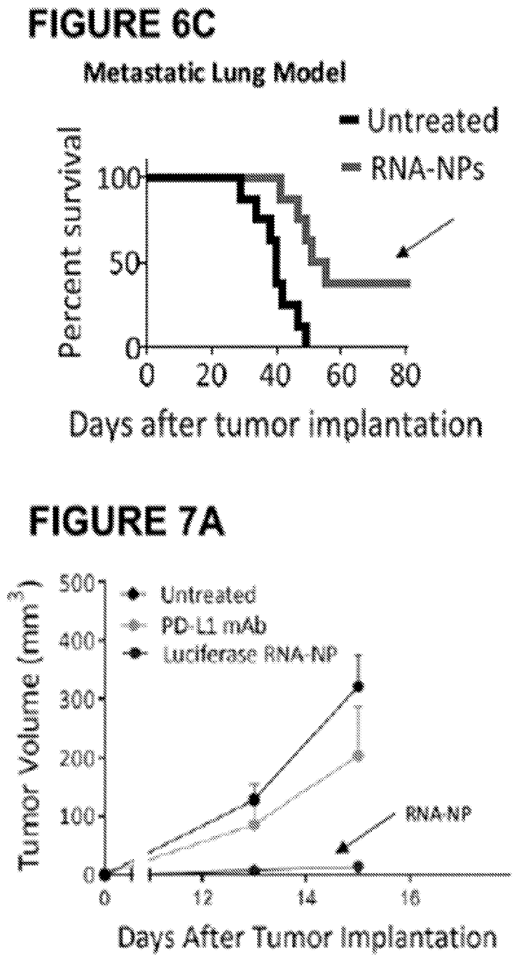

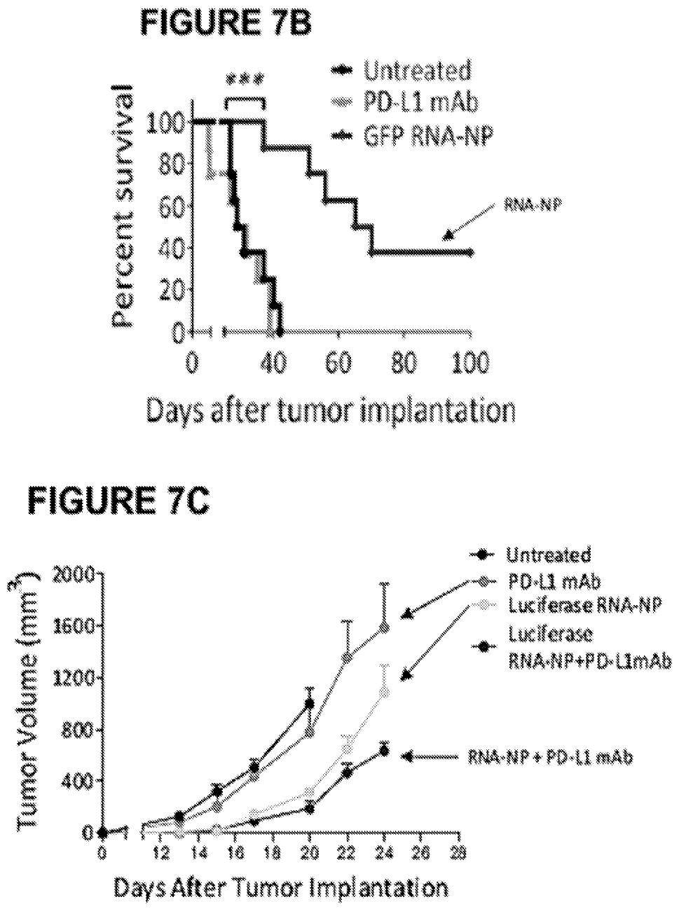

This Example demonstrates that ML RNA-NPs of the instant disclosure mediate anti-tumor immune responses against immunologically “cold” tumors, i.e. tumors which did not respond to ICIs. As demonstrated in FIGS. 6A-6C, administration of the ML RNA-NPs of the disclosure with an immune checkpoint inhibitor (here, anti-PD-L1 antibody) resulted in reduced tumor volumes in a melanoma model compared with administration of RNA-NP alone and checkpoint inhibitor alone. Administration of ML RNA-NPs also resulted in enhanced subject survival in a sarcoma model and a metastatic lung model. The data establish that ML RNA-NPs reprogram immunologically “cold” tumors, as well as demonstrate the effectiveness of the ML RNA-NPs over a range of cancers and tumor types.

### Example 8

This Example describes studies to elucidate the impact of systemic administration of the nanoparticle composition of the disclosure on CAR T cell priming.

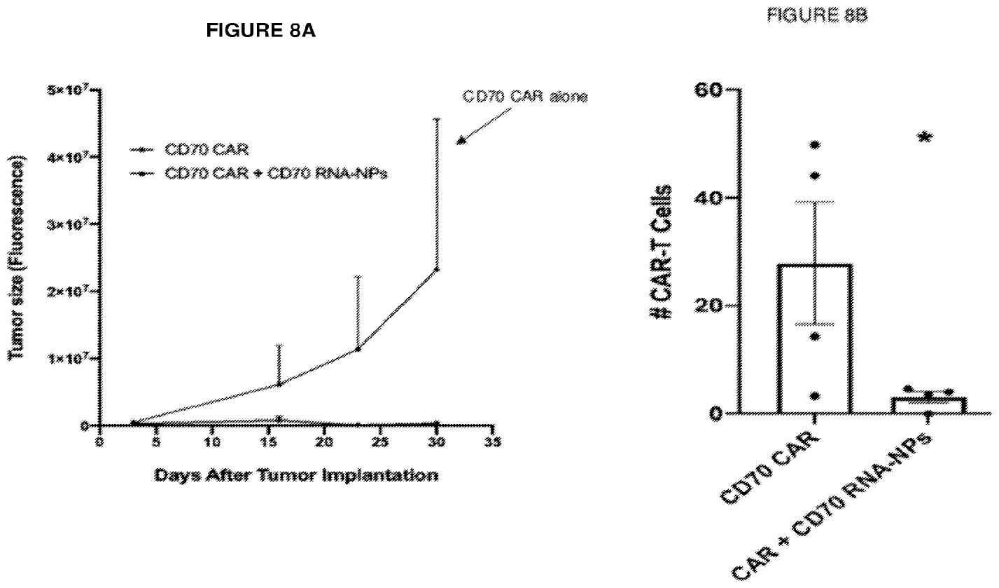

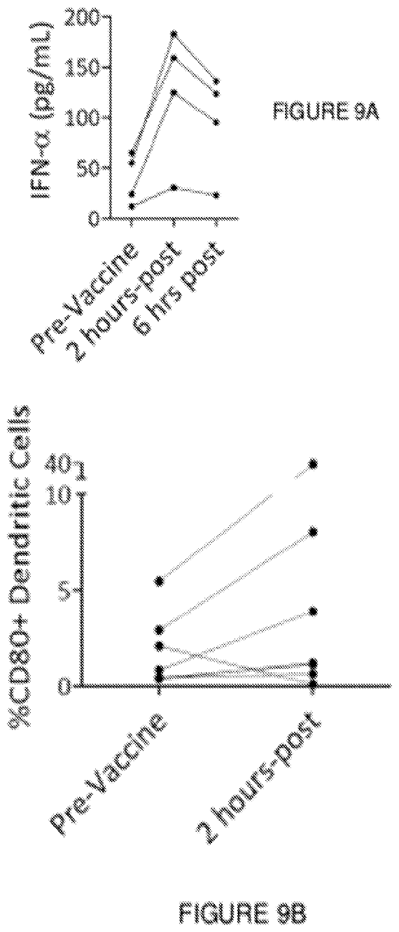

CD70 and GD2 are surface antigens expressed in osteosarcoma (OSA). A CD70-CAR T platform demonstrates promising antitumor activity against CD70-expressing solid tumors. Jin et al., Nat Commun., 10(1):4016 (2019). In in vitro experiments, tumor-specific killing against CD70-KR158 and CD70-K7M2 OSA tumor cells was observed. Additionally, a GD2-CAR T cell construct was generated which targets GD2-expressing OSA cell lines (K7M2 and MOS-J). In follow-up experiments, it was shown that CAR T cells mediate substantial regression of established solid tumors when combined with RNA-NPs encoding for surface targets (i.e., CD70) that nicely correlates with CAR T mobilization into tissues (FIG. 8A).

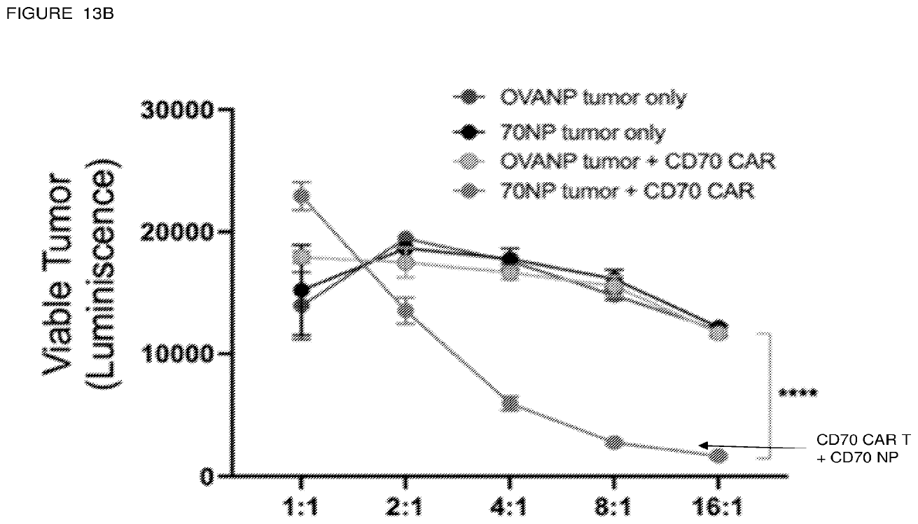

To determine the effects of NP-activated APCs on CAR specific T cells (administered one week after implantation), K7M2 bearing mice (inoculated i.v. with pulmonary OSA metastasis) were vaccinated with purified tumor mRNA (derived from CD70 expressing K7M2) encapsulated into FITC-labeled NPs as previously described (Sayour et al. Oncoimmunology 2016: e1256527; doi: 10.1080/2162402X.2016.1256527). RNA-NPs composed from 375 μg of lipid-NP described herein with 25 μg of tumor mRNA (from K7M2) (versus NPs alone or control RNA-NP) were administered iv weekly (×3) beginning 24 hours after CAR T cell administration. CAR transgene expression was assessed from peripheral blood. One week after the last vaccine, relevant FITC+ DCs (CD11c+CD86+ MHC+ cells) were sorted (FACSort; BD Aria II) from spleens, tumor draining lymph nodes (tdLNs) and OSA tumors. RNA-NP transfected DCs were then cultured with CAR T cells, and T cells were assessed for proliferation, phenotype (effector vs central memory), function, and cytotoxicity. T cell proliferation was assessed via CFSE dilution by flow cytometry. Phenotype for effector/central memory cells was determined through differential staining for CD44 and CD62L. These T cells were re-stimulated for a total of two cycles before supernatants are harvested for detection of Th1 cytokines (i.e., IL-2, TNF-α, and IFN-γ) by bead array (BD). To determine antigen specificity, T cells were incubated in the presence of K7M2 (transfected with GFP or luciferase, expressing surface target) or control tumor (B16F10-GFP, non-surface target expressing K7M2) and assessed for cytotoxic killing. The amount of GFP or luciferase in each co-culture, as a surrogate for living tumor cells, was quantitatively measured by flow cytometry and bioluminescence. Representative results of this type of assay are illustrated in FIG. 13A, which illustrates transduction of spleen and liver cells using RNA-NP, and FIG. 13B, which corresponds to a study using tumor cells, RNA-NP, and CAR T-cells.

Recruitment of DCs to tumors is typically associated with a regulatory phenotype characterized by increased IDO, FoxP3+ Tregs and secretion of immunoregulatory cytokines. To determine intratumoral effects of DCs, CAR T cells with or without tumor mRNA-NPs (versus NPs alone or control RNA-NPs) are administered to K7M2 bearing IFN-γ reporter mice once weekly (×3) with and without DC depleting mAbs (Bioxcell). Activated and regulatory T cells are examined over time in the intratumoral microenvironment at serial time points (6 h, 1 d, 7 d, and 21 d). Effector T cells may be characterized as previously described (Sayour et al. Nano Lett, 18(10):6195-206 (2018)) and Tregs are phenotyped through expression of FoxP3, CD25, and CD4. DCs from non-depleted animals are FACSorted and phenotyped for expression of multiplex cytokines, chemokines (i.e., IL-2, TNF-alpha, IFN-I/II, MIP-1-alpha/beta), activation markers (i.e., CD80, CD86, CD40), cytolytic markers (i.e., TRAIL, granzyme b) and regulatory markers (i.e., IL-10, TGF-β, IDO). Immunophenotypic changes by tumor cells are also assessed (i.e., MHC-I, PD-L1, SIRPα).

### Example 9

This Example describes studies to examine the mechanistic underpinnings to CAR T cell trafficking and persistence in the OSA tumor microenvironment (TME).

RNA-NPs upregulate LFA-1 and CCR2 on T cells in an interferon-I dependent manner. These findings correlate with massive mobilization of lymphocytes and monocytes out of peripheral blood in large animals that received RNA vaccine. Since high-grade OSAs express increased levels of CCL2 compared with low-grade OSAs, this approach may drive CAR T cell therapy into the OSA TME leading to enhanced anti-tumor activity and persistence.

The following study serves to examine the chemokine receptor, S1P1, and VLA-4/LFA-1 expression profile of CAR T cells before and after RNA-NP vaccination. The methodology allows assessment of RNA-NPs' effects on CAR T cell trafficking molecules, including sphingosine-1-phosphate receptor 1 (S1P1), which is necessary for T cell egress from lymphoid organs, and integrins (i.e., VLA-4, LFA-1) necessary for T cell passage into the TME. K7M2 bearing IFN-γ reporter mice are administered CAR T cells alone or CAR T cells in combination with RNA-NPs. One week after the last vaccine, recipient mice are humanely euthanized (with CO2) and spleens, tdLNs, bone marrow, and tumors are harvested. Organs are digested, and CAR T cells from spleens, lymph nodes, bone marrow and tumors are identified by surface expression of target antigen and by differential staining for effector and central memory T cells (i.e., CD62L and CD44 markers) at serial time points (7, 14 and 21 days). Th1-associated chemokine receptors (i.e., CCR2, CCR5, CCR7 and CXCR3), S1P1 expression, VLA-4, and LFA-1 expression (ebioscience) from CD4 and CD8 CAR T cells are assessed by multi-parameter flow cytometry and IHC.

The following study serves to examine the effects of RNA-NP on in vitro and in vivo migration of CAR T cells and persistence in the OSA TME. In vitro: K7M2 tumor bearing naïve, LFA-1, CCL2 or CCR2 KO animals (B6 transgenic, Jackson) are administered CAR T cells with or without RNA-NPs (versus NPs alone or control RNA-NPs) weekly (×3). One week after the last vaccine, T cells are FACSorted via a BD Aria II Cell Sorter. These T cells may be assessed for migratory capacity in transwell assays (Thermo-Fisher). Briefly, T cells are placed in the upper layer of a cell culture insert with a permeable membrane in between a layer of K7M2-GFP tumor cells. Migration is assessed by number of cells that shift between the layers. T cells are plated in T cell media with and without IL-2 (1 microgram/mL) at a concentration of 4×10⁶ per mL for co-culture with tumor cells (4×10⁶/mL) (×48 hrs) before determination of IFN-γ by ELISA. The amount of GFP in each co-culture, as a surrogate for living tumor cells, is quantitatively measured by flow cytometric analysis and bioluminescence. In vivo: K7M2 bearing IFN-γ reporter mice, or IFN-γ reporter mice receiving LFA-1 or CCL2 blocking mAbs (Bioxcell) are administered CAR T cells with or without RNA-NPs (versus NPs alone or control RNA-NPs) weekly (×3). In vivo passage into the TME is assessed from percentage and absolute numbers of CAR T cells in OSA tumors (relative to spleen, lymph nodes, bone marrow) at serial time points (5d, 10d, 15d, 20d from 1st vaccine). Antigen specific T cell cytotoxicity assays are performed as described above.

### Example 10

This Example describes studies to examine the immunologic activity of CAR T cells with and without systemic vaccination in a large animal OSA model.

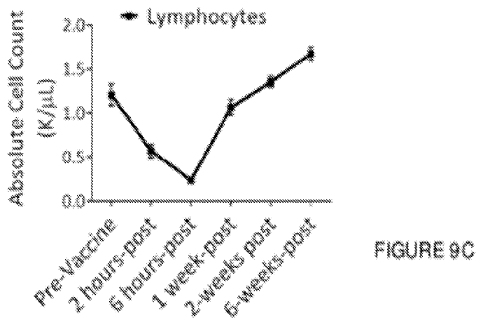

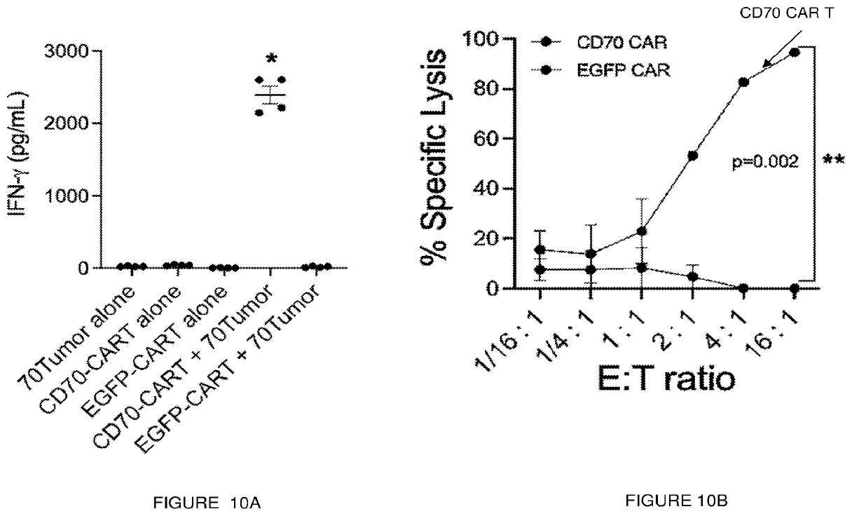

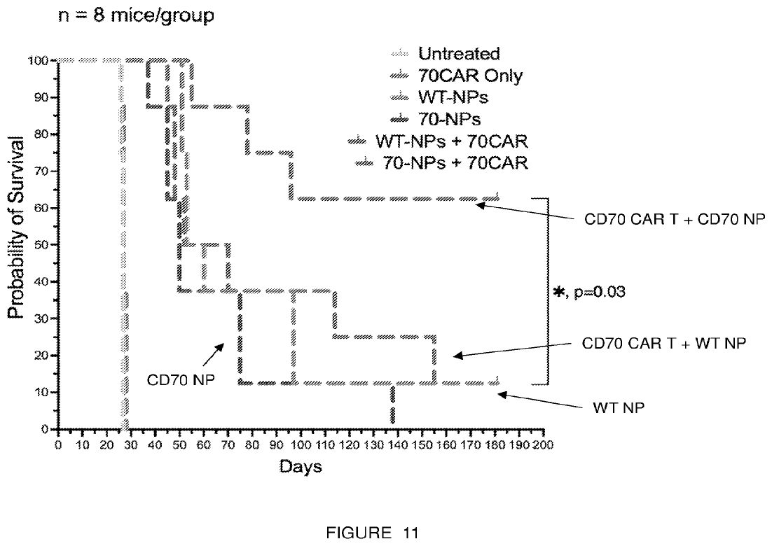

Two canines with OSA (which naturally expressed CD70) were treated with RNA-NPs, and transformed canine T cells with CARs were generated. Remarkably, one patient with OSA continues to survive. In canines with OSA, it was observed that, within a few hours after administration, tumor specific RNA-NPs elicited margination of peripheral blood mononuclear cells, which increased in the subsequent days and weeks post-treatment (FIGS. 9A-C), suggesting that RNA-NPs mediate lymphoid honing of immune cell populations before egress. RNA-NPs also elicited increases in: 1) serum IFN-α that spiked at 2 hrs; 2) CD86, PD-L1 and MHCII on CD11c+ peripheral blood cells (demonstrating activation of peripheral DCs); and 3) the percentage of activated CD8+ T cells.

The following study examines combination therapy of RNA-NPs and CAR T cells in a canine model of OSA. Routine standard of care for canines with metastatic OSA involves palliation (no biopsies) and is uniformly fatal. Canines with suspicion for metastatic OS (based on imaging) will be enrolled. Canines will then undergo a screening CT guided biopsy for confirmation of disease by histopathology, and screening for GD2 or CD70 expression. If patient meets eligibility, we will manufacture GD2 or CD70 CAR T cells and administer these (1×10⁷ cells/kg) in conjunction with tumor specific RNA-NPs. Validation of personalized tumor mRNA will be determined based on RNA quality, concentration and integrity by gel electrophoresis, nanodrop spectrophotometry and bioanalysis. Two weeks after biopsy and peripheral blood mononuclear cells (PBMCs) collection, CAR T cells alone or CAR T cells and RNA-NPs (1 h apart) will be given to enrolled canines. Peripheral labs and serum cytokines (i.e., IFI-I, IL-6, TNF-α) from dogs are monitored at diagnosis, immediately before each bi-weekly RNA-NP vaccine (×3) and during monthly post-treatment follow-ups.

The following study examines peripheral DC and CAR T cell activation phenotype in canines diagnosed with OSA.

Identifying biologic correlates for immunotherapeutic response has been a significant challenge that has stymied the development of new immunotherapies. DC and CAR T cell phenotypes requisite for immunologic response or resistance may be examined as follows. Briefly, at diagnosis, and immediately preceding each vaccination, 10-50 mLs of peripheral canine blood is drawn into vacutainer tubes. PBMCs are separated by density gradient centrifugation via Ficoll. DCs are assessed weekly from PBMCs for determination of activation markers (i.e., CD80, CD86, and MHCII on CD11c+ cells). CAR transgene expression is assessed. T cell lymphocytes are analyzed for CD3, CD4, CD8, CCR2, CD69, LFA-1 and PD-1 surface expression and intracellular staining is performed for FoxP3 and IFN-γ. Analysis is monitored using multi-color flow cytometry. To assess efficacy, tumor growth is determined based on CT imaging (at 2 and 4 weeks post RNA-NPs, and every 3 months thereafter). Immunologic escape mechanisms are examined in tumors obtained via necropsy (i.e., expression of checkpoint ligands, IDO, downregulation of MHC class I) and within the OSA TME (i.e., MDSCs, Tregs, and TAMs). Escape mechanisms in tumors and within the TME is conducted by multi-parameter flow cytometry (LSR, BD Bioscience).

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the disclosure (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. If aspects of the invention are described as “comprising” a feature, embodiments also are contemplated “consisting of” or “consisting essentially of” the feature.

Recitation of ranges of values herein are merely intended to serve as a shorthand method of referring individually to each separate value falling within the range and each endpoint, unless otherwise indicated herein, and each separate value and endpoint is incorporated into the specification as if it were individually recited herein. Other than in the operating examples, or where otherwise indicated, all numbers expressing quantities of ingredients or reaction conditions used herein should be understood as modified in all instances by the term “about” as that term would be interpreted by the person skilled in the relevant art.

All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

Preferred embodiments of this disclosure are described herein, including the best mode known to the inventors for carrying out the disclosure. Variations of those preferred embodiments may become apparent to those of ordinary skill in the art upon reading the foregoing description. The inventors expect skilled artisans to employ such variations as appropriate, and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

What is claimed:

1. A method of preconditioning a subject for chimeric antigen receptor (CAR) T cell therapy, the method comprising administering to the subject a first composition comprising a nanoparticle comprising a positively-charged surface and an interior comprising (i) a core and (ii) at least two nucleic acid layers, wherein each nucleic acid layer is positioned between a cationic lipid bilayer, at least one day prior to administering CAR T cell therapy to the subject.

2. The method of claim 1, wherein the composition is administered between two and 14 days prior to administering the CAR T cell therapy to the subject.

3. The method of claim 1 or claim 2, wherein the composition is administered about five to about eight days prior to administering the CAR T cell therapy to the subject.

4. The method of any one of claims 1-3, wherein the subject is not administered lymphodepletion therapy within 21 days prior to administration of the CAR T cell therapy.

5. The method of any one of claims 1-4, wherein the subject is suffering from a solid tumor.

6. The method of any one of claims 1-5, wherein the subject is suffering from an immune checkpoint inhibitor (ICI)-resistant malignancy.

7. The method of any one of claims 1-5, wherein the subject is suffering from a refractory malignancy.

8. The method of any one of claims 1-7, wherein the subject is suffering from a malignant brain tumor.

9. The method of claim 8, wherein the malignant brain tumor is a glioblastoma, medulloblastoma, diffuse intrinsic pontine glioma, or a peripheral tumor with metastatic infiltration into the central nervous system.

10. The method of claim 5, wherein the subject is suffering from recurrent or metastatic osteosarcoma.

11. The method of any one of claims 1-10, wherein the nanoparticle comprises at least three nucleic acid layers, each of which is positioned between a cationic lipid bilayer.

12. The method of any one of claims 1-11, wherein the outermost layer of the nanoparticle comprises a cationic lipid bilayer.

13. The method of any one of claims 1-12, wherein the surface comprises a plurality of hydrophilic moieties of the cationic lipid of the cationic lipid bilayer.

14. The method of any one of claims 1-13, wherein the core comprises a cationic lipid bilayer.

15. The method of any one of claims 1-14, wherein the core comprises less than about 0.5 wt % nucleic acid.

16. The method of any one of claims 1-15, the nanoparticle comprises a zeta potential of about 40 mV to about 60 mV

17. The method of claim 16, wherein the nanoparticle comprises a zeta potential of about 45 mV to about 55 mV.

18. The method of any one of claims 1-17, wherein the cationic lipid is DOTAP or DOTMA.

19. The method of any one of claims 1-18, wherein the nucleic acid is mRNA.

20. The method of claim 19, wherein the mRNA is tumor mRNA.

21. The method of claim 20, wherein the mRNA is in vitro transcribed mRNA wherein the in vitro transcription template is cDNA made from RNA extracted from a tumor cell.

22. The method of claim 19, wherein the mRNA does not encode a tumor antigen targeted by the CAR T cell.

23. The method of claim 19, wherein the mRNA is not tumor mRNA.

24. The method of any one of claims 1-23, wherein the nanoparticle does not comprise a neutral lipid.

25. The method of any one of claims 1-24, further comprising administering a second composition comprising a nanoparticle comprising a positively-charged surface and an interior comprising (i) a core and (ii) at least two nucleic acid layers, wherein each nucleic acid layer is positioned between a cationic lipid bilayer, after administration of the CAR T cell therapy.

26. The method of claim 25, wherein the nucleic acid of the second composition is tumor mRNA.

27. The method of claim 26, wherein the mRNA is in vitro transcribed mRNA wherein the in vitro transcription template is cDNA made from RNA extracted from a tumor cell.

28. A method of treating a solid tumor in a subject, the method comprising administering to a subject comprising a surface antigen negative solid tumor a first composition comprising a nanoparticle comprising a positively-charged surface and an interior comprising (i) a core and (ii) at least two nucleic acid layers, wherein each nucleic acid layer is positioned between a cationic lipid bilayer and the nucleic acid encodes the surface antigen, and a second composition comprising a T cell expressing a chimeric antigen receptor (CAR) that targets the surface antigen.

29. The method of claim 28, wherein the first composition is administered at least one day prior the second composition.

30. The method of claim 28, wherein the first composition is administered at least once between two and 14 days prior to administering the second composition to the subject.

31. The method of any one of claims 28-30, wherein the subject is not administered lymphodepletion therapy within 21 days prior to administration of the CAR T cell therapy.

32. The method of any one of claims 28-31, where in the solid tumor is present in lung, liver, bone, spleen, or lymph node.

33. The method of claim 32, wherein the subject is suffering from recurrent or metastatic osteosarcoma.

34. The method of any one of claims 28-33, wherein the nanoparticle comprises at least three nucleic acid layers, each of which is positioned between a cationic lipid bilayer.

35. The method of any one of claims 28-34, wherein the outermost layer of the nanoparticle comprises a cationic lipid bilayer.

36. The method of any one of claims 28-35, wherein the surface comprises a plurality of hydrophilic moieties of the cationic lipid of the cationic lipid bilayer.

37. The method of any one of claims 28-36, wherein the core comprises a cationic lipid bilayer.

38. The method of any one of claims 28-37, wherein the core comprises less than about 0.5 wt % nucleic acid.

39. The method of any one of claims 28-38, the nanoparticle comprises a zeta potential of about 40 mV to about 60 mV

40. The method of claim 39, wherein the nanoparticle comprises a zeta potential of about 45 mV to about 55 mV.

41. The method of any one of claims 28-40, wherein the cationic lipid is DOTAP or DOTMA.

42. The method of any one of claims 28-41, wherein the nucleic acid is mRNA.

43. The method of any one of claims 28-42, wherein the nanoparticle does not comprise a neutral lipid.

44. The method of any one of claims 28-43, comprising administering the first composition after administration of the second composition.

45. The method of any one of claims 28-44, further comprising administering a third composition comprising a nanoparticle comprising a positively-charged surface and an interior comprising (i) a core and (ii) at least two nucleic acid layers, wherein each nucleic acid layer is positioned between a cationic lipid bilayer and the nucleic acid does not encode the surface antigen.

46. The method of claim 45, wherein the nucleic acid of the third composition is tumor mRNA.

47. The method of claim 45, wherein the nucleic acid of the third composition is not tumor RNA.

48. The method of any one of claims 28-47, wherein the surface antigen is CD70.


SEQUENCE LISTING (1 sequence)

SEQ ID NO: 1 - DNA, 1100 bp, Artificial Sequence
  Synthetic
tggcaagaccccacccctggtggctgtgtgtgctgggcacactggtcggactgagcgccacccctgcccctaagagctgccccgagagacactactgggctcagggcaagctgtgctgccagatgtgcgagcccggcaccttcctggtgaaagactgcgaccagcaccggaaggccgcccagtgcgatccttgcatccccggcgtgtccttcagccccgaccaccacaccagaccccactgcgagagctgccggcattgcaactctggcctgctggtccgcaactgcaccatcaccgccaacgccgagtgcgcctgcagaaacggctggcagtgccgggacaaagaatgcaccgagtgcgaccctctgcccaaccccagcctgaccgccagaagcagccaggctctgagccctcaccctcagcccacccatctgccctacgtgtccgagatgctggaagcccggacagccggccacatgcagaccctggccgacttcagacagctgcccgccagaaccctgagcacccactggcctccccagcggagcctgtgcagcagcgacttcatccggatcctggtgatcttcagcggcatgttcctggtgttcaccctggctggcgccctgttcctgcacaagcggggcagaaagaagctgctgtacatcttcaagcagcccttcatgcggcccgtgcagaccacccaggaagaggacggctgcagctgccggttccccgaggaagaggaaggcggctgcgagctgagagtgaagttcagcagaagcgccgacgcccctgcctaccagcagggccagaaccagctgtacaacgagctgaacctgggcagacgggaagagtacgacgtgctggacaagcggagaggccgggaccctgagatgggcggcaagccccagaggcggaagaaccctcaggaaggcctgtataacgaactgcagaaagacaagatggccgaggcctacagcgagatcggcatgaagggcgagcggcggagaggcaagggccacgatggcctgtaccagggcctgagcaccgccaccaaggacacctacgacgccctgcacatgcaggctctgcctccaaga